(19) United States
(12) Patent Application Publication
(10) Pub. No.: US 2026/0090920 A1
(43) Pub. Date: Apr. 2, 2026

(54) METHOD FOR FORMING A CELLULOSIC TAMPON APPLICATOR

(71) Applicant: THE PROCTER & GAMBLE COMPANY, Cincinnati, OH (US)

(72) Inventors: Evan Joseph DURLING, West Chester, OH (US); Kevin Charles STRONG, Loveland, OH (US); Khalid QURESHI, Mason, OH (US); Ryo MINOGUCHI, Cincinnati, OH (US)

(21) Appl. No.: 19/345,643

(22) Filed: Sep. 30, 2025

Related U.S. Application Data
(60) Provisional application No. 63/701,856, filed on Oct. 1, 2024.

(51) Int. Cl. A61F 13/20; D21J 3/04

(52) U.S. Cl. CPC A61F 13/2097; A61F 13/2051; D21J 3/04

(57) ABSTRACT

A method is presented for forming a tampon applicator. The method includes providing an applicator comprising an outer member and an inner member. Each of the outer member and the inner member comprise cellulosic material. The method further includes loading the outer member into a compression device. The method further includes compressing the outer member to form a grip region. The grip region comprises greater than four faces that are circumferentially formed about the outer member. The method further includes unloading the outer member comprising the grip region and a barrel region from the compression device.

### CROSS REFERENCE TO RELATED APPLICATIONS

This application claims the benefit, under 35 U.S.C. § 119(e), to U.S. Provisional Application No. 63/701,856, filed Oct. 1, 2024, the entire disclosure of which is fully incorporated by reference herein.

### FIELD

The present disclosure relates to tampon applicators, and more particularly, to a method for forming tampon applicators made of cellulosic material.

### BACKGROUND

Consumer products or portions thereof that have components derived from petroleum (such as but not limited to plastics), are currently becoming subject to increased regulation by governments in a number of countries, and growing disfavor in some segments of the consumer market. Some reasons for this relate generally to the consumer perception of certain materials having a relatively adverse impact on the environment.

Tampons have been used for many years to manage menstrual discharge. Many consumers prefer tampons provided inside disposable applicators (rather than tampons configured to be inserted via a finger (“digitally”) without use of an applicator). The typical tampon applicator is configured to house and protect the new tampon from deformation and/or contamination prior to use, and then to facilitate application of the tampon when its use is desired.

Currently, many tampon applicators or parts thereof are formed of thermoplastics such as polyethylene. The properties of thermoplastics and known associated forming/molding technologies enable a manufacturer to efficiently make an applicator having relatively sophisticated features, which may be provided for purposes of functionality, convenience, comfort during use, ergonomics and/or aesthetic appeal, thereby making it attractive and convenient to consumers. FIG. 1A depicts a conventional plastic applicator 10 with a barrel region 12 having an external diameter 13, a plunger 30 having an external diameter 31 and a grip region 18 positioned between the barrel region 12 and plunger 30. As shown in FIG. 1A, the external diameter 31 of the plunger 30 is noticeably less than the external diameter 13 of the barrel region 12.

Other types of tampon applicators currently marketed are formed of paper or cardboard. While such applicators reduce reliance on and use of materials derived from petroleum as compared to plastic applicators, currently, these cardboard applicators do not provide the same functionality, comfort during use, ergonomics and/or aesthetic appeal as plastic applicators. FIG. 1B depicts a conventional cardboard applicator 10′ including a barrel region 12′ having an external diameter 13′, a plunger 30′ having an external diameter 31′ and a grip region 18′ between the barrel region 12′ and the plunger 30′. Unlike the plastic applicator 10 of FIG. 1A, the external diameter 31′ of the plunger 30′ is relatively close the external diameter 13′ of the barrel region 12′. FIG. 1C depicts another conventional cardboard applicator 10′ where the grip region 18′ includes four impressions 40′ which are spaced apart by the outer surface 42′ of the grip region 18′.

Accordingly, there remain opportunities for improvements in methods for forming carboard applicators so to enhance their design and functionality such that carboard applicators more closely emulate their plastic applicator counterparts.

### SUMMARY

The inventors of the present invention recognized various advantages of the conventional plastic applicator that are absent in conventional paper or cardboard applicators. These include features of the plastic applicators which facilitate functionality, convenience, comfort during use, ergonomics and aesthetic appeal. The inventors also recognized that recent environmental regulations in various geographical territories that promote the use of cardboard material over plastic material will likely cause a shift in consumer demand towards cardboard applicators. Thus, the inventors developed a method for forming an improved cardboard applicator herein, which achieves many of the advantages of plastic applicators that are absent in conventional cardboard applicators. In order to develop this method for forming the improved cardboard applicator, the inventors necessarily derived method steps that overcame various design obstacles not encountered in manufacturing plastic applicators.

In some embodiments, a method is presented for forming a tampon applicator. The method includes providing an applicator comprising an outer member and an inner member. Each of the outer member and the inner member comprise cellulosic material. The method further includes loading the outer member into a compression device. The method further includes compressing the outer member to form a grip region. The grip region comprises greater than four faces that are circumferentially formed about the outer member. The method further includes unloading the outer member comprising the grip region and a barrel region from the compression device.

In some embodiments, a method is presented for forming a tampon applicator. The method includes providing an outer member comprising cellulosic material. The method also includes loading the outer member into a radial compression device. The method also includes radially compressing the outer member to form a grip region. The method further includes unloading the outer member comprising the grip region and a barrel region from the radial compression device.

In some embodiments, a method is presented that is similar to the aforementioned embodiments except that the outer member includes two or more layers of cellulosic material that are formed by at least one of spirally winding or convolutely winding the two or more layers.

These and other features, aspects, and advantages of various embodiments will become better understood with reference to the following description, figures, and claims.

### BRIEF DESCRIPTION OF THE DRAWINGS

FIG. 1A is a front view of a conventional plastic applicator including a barrel region and a plunger.

FIG. 1B is a front view of a conventional cardboard applicator including a barrel region and a plunger.

FIG. 1C is a front view of a conventional cardboard applicator.

FIG. 2A is a perspective view of a tampon product with an applicator including a barrel region and a plunger.

FIG. 2B is a longitudinal side view of the tampon product of FIG. 2A taken along the line 2B-2B.

FIG. 2C is a longitudinal side view of a plunger of the tampon product of FIG. 2A with a flange at a second end region thereof.

FIG. 2D is a perspective view of a plunger of the tampon product with a flange at a first end region and second end region thereof.

FIG. 3A is a perspective view of a machine used to form circumferential faces in the grip region.

FIG. 3B is a perspective view of a plurality of forming members of the machine of FIG. 3A that move radially inward to form the circumferential faces in the grip region of the applicator.

FIG. 3C is an end view of a stabilizing rod positioned within an interior portion of the applicator prior to the forming members of FIG. 3B forming the circumferential faces.

FIG. 3D is a perspective view of a forming member of the machine and a stabilizing rod comprising a plurality of interconnected members.

FIG. 3E is an end view of the forming members of FIG. 3B radially compressing the applicator and pinching the applicator between the forming members to form ribs.

FIG. 3F is an end view of an odd number of forming members of a machine that move radially inward to form the circumferential faces in the grip region of the applicator.

FIG. 3G is an end view of the forming members of FIG. 3B radially compressing the applicator with no space between adjacent forming members.

FIGS. 3H and 3I are side views of the applicator respectively before and after the compression with the forming members in FIG. 3G.

FIG. 3J is a top perspective view of a forming member secured to the machine of FIG. 3A having different profiles at different respective ends thereof.

FIG. 3K is a side view of a plurality of grip regions formed in the applicator using the first profile on the first end of the forming member of FIG. 3J.

FIG. 3L is a side view of a plurality of grip regions formed in the applicator using the second profile on the second end of the forming member of FIG. 3J.

FIGS. 3M through 3P are various views showing a stabilizing rod and a forming member.

FIG. 3Q is a side view of a plurality of grip regions formed in the applicator using the forming member and stabilizing rod of FIGS. 3M through 3P.

FIG. 3R is an applicator parent roll with a plurality of spaced apart grip regions formed along a length of the applicator parent roll.

FIGS. 4A through 4C are perspective and top views of a plurality of circumferential faces and ribs therebetween formed in the grip region.

FIG. 4D is a cross-sectional view of the grip region of FIG. 4A taken along the line 4D-4D.

FIG. 4E is a cross-sectional view of a rib of the cross-sectional view of the grip region of FIG. 4D.

FIG. 4F is a cross-sectional view of a rib of the cross-sectional view of the grip region.

FIG. 4G is a cross-sectional view of the grip region of FIG. 4C taken along the line 4G-4G.

FIG. 4H is a cross-sectional view of the grip region of FIG. 4A taken along the line 4H-4H.

FIGS. 5A through 5C are perspective views of an end region of the barrel region of the applicator of FIG. 2A.

FIGS. 5D through 5F are perspective views of various curling tools that are used to compress the end regions of FIGS. 5A through 5C.

FIGS. 5G through 5I are perspective views of a forward end region of the barrel region.

FIGS. 5J through 5L are perspective views of various pleating tools that are used to compress the forward end regions of FIGS. 5G through 5I.

FIGS. 6A through 6C are side views of an applicator including a barrel region and a plunger with circumferential faces formed in both the barrel region and the plunger.

FIG. 7A is a cross-sectional side view of a plurality of material layers that define the applicator.

FIG. 7B is a side view of an applicator including a spirally wound barrel region.

FIG. 7C is a side view of an applicator including a convolutely wound barrel region.

FIG. 7D is a cross-sectional side view of a plurality of material layers that define the applicator.

FIG. 8 is a flowchart depicting one or more steps of a method for forming a tampon applicator.

### DETAILED DESCRIPTION

The following term explanations may be useful in understanding the present disclosure.

As used herein the term “tampon” refers to any type of absorbent structure which is configured to be inserted into the vaginal cavity for the interception and absorption of fluid therefrom. Typically, a tampon includes a pledget structure including a quantity of absorbent material, often absorbent fibrous material, which pledget structure has been bunched, folded and/or compressed in one or more lateral/radial directions, the longitudinal direction, or both, via application of pressure, heat and/or moisture control, in order to provide a formed tampon having a size, shape (typically approximately cylindrical) and stability of form to facilitate insertion into the vagina. A tampon which has been so formed is referred to herein has a “self-sustaining” form. The degree of compression, heat and moisture control applied to the pledget is sufficient such that in the subsequent absence of the external forces and absence of substantial contact with moisture, the pledget will tend to retain its general formed shape and size. It will be understood by persons of ordinary skill in the art that this self-sustaining form typically does not persist following insertion of the tampon. Once the tampon is inserted and begins to contact and absorb fluid, the pledget will swell with absorbed fluid and lose its self-sustaining form.

As used herein the terms “pledget” or “tampon pledget” are intended to be interchangeable and refer to a structure including absorbent material configured to perform the primary function of the tampon, absorption of menstrual fluid. A tampon pledget is sometimes referred to as a tampon blank, or a softwind, and the term “pledget” is intended to include structures designated by such terms as well.

As used herein “vaginal cavity” refers to the internal space within the genitalia of the human female, located between the introitus of the vagina (sometimes referred to as the sphincter of the vagina) and the cervix.

With respect to a tampon and an applicator, the “longitudinal” direction is the ordinary general direction of ejection from an applicator; and also corresponds with the ordinary general direction of insertion of a tampon and applicator into, and their withdrawal from, the vaginal cavity in normal use. For a completely manufactured, pre-use tampon that has a pledget with a generally cylindrical or capsule-shaped self-sustaining form, the longitudinal axis of the form lies generally or approximately along the longitudinal direction. A “radial” or “circumferential” direction is a direction perpendicular to the longitudinal direction. The “circumferential” direction is perpendicular to the longitudinal direction and the radial direction, and perpendicular to the z direction (defined below). Unless otherwise specified, references to “length” herein refer to a dimension along the longitudinal direction; references to “width” herein refer to a dimension along the circumferential direction.

With respect to a tampon and an applicator, the term “forward” refers to a longitudinal direction of movement during normal insertion of the tampon and/or applicator by a user, and refers to portions of the tampon or applicator that lie closer to and/or enter the vaginal cavity earlier than other portions during normal insertion. Conversely, the term “rearward” refers to a longitudinal direction of movement during normal withdrawal of the tampon and/or applicator by a user, and refers to portions of the tampon or applicator that lie farther from and/or exit the vaginal cavity earlier than other portions during normal withdrawal.

A “nonwoven,” “nonwoven web,” “nonwoven web material,” or “nonwoven fabric” is a cloth-like web material (or portion or section thereof) formed predominantly of fibers that are neither knitted nor woven, but rather, laid down and accumulated to a desired basis weight, then consolidated and held together to form a web, via one or any combination of calendering, thermal and/or compression bonding, bonding via use of a binder, heating (via, e.g., heated air driven through an accumulation of fibers) or hydroentangling (spunlace). The predominant fibers may be natural fibers harvested from plant material (e.g., cotton) (but excluding tree wood pulp), semi-synthetic (e.g., rayon, lyocell, viscose), or synthetic (e.g., fibers spun from molten thermoplastic polymer resin(s)), or any combination thereof. Herein, a skin- or membrane-like film (e.g., extruded or otherwise formed from polymer resin(s)) is not deemed a nonwoven. Herein, a paper tissue product, paper product, or paperboard or cardboard product, formed via wetlaying and predominantly constituted of tree wood pulp, is not deemed a nonwoven.

“Opened configuration,” with respect to a tampon, means the configuration of the pledget prior to the time it is compressed and formed into a self-sustaining form during manufacture, or in the case of a finished product, after it is completely ejected from an applicator (if present) and/or allowed and/or caused by any suitable technique to open and substantially re-assume its pre-compression shape and size.

“Paper” or “Cardboard” means a material in web or sheet form, formed predominantly of cellulose fibers, for example, wood pulp fibers, which have been suspended in a slurry, which is then poured onto a moving mesh belt, drained of water, and subsequently dried over drying rollers, and in many examples, finished via calendering. In the resulting web or sheet product the cellulose fibers are interlaid and randomly oriented. The paper or cardboard herein may also be substantially void of synthetic materials as defined herein, such as fibers, films, and/or adhesives. It is also to be appreciated that paper or cardboard may be formed by pulp molding.

“Predominant,” and forms thereof, when used to characterize a quantity of a constituent present in a composition, means that a majority of the weight of the material is constituted by the constituent.

“Withdrawal cord” refers to any section of string, yarn, cord, ribbon, strip material or other flexible/pliable elongate structure typically (although not necessarily) formed of fibrous material, attached to and/or extending from a tampon pledget and trailing from its rearward end. A withdrawal cord of sufficient length may be provided with a tampon for the purpose of providing a relatively thin and flexible trailing member of sufficient length to allow for a portion thereof to trail and remain outside of the introitus following full insertion of the tampon, which the user may easily grasp and pull to withdraw the tampon from her body following a desired duration of use.

As used herein, the term “joined” encompasses configurations whereby an element is directly secured to another element by affixing the element directly to the other element, and configurations whereby an element is indirectly secured to another element by affixing the element to intermediate member(s) which in turn are affixed to the other element.

With respect to a paper sheet or web product, when laid out flat on a horizontal planar surface, the “z direction” is a direction orthogonal to the horizontal planar surface and is the direction along which caliper or thickness of the sheet or web (prior to rolling into paper tube product) would be measured.

“Synthetic materials” are those that are chemically modified such that the polymerization was not naturally occurring. Synthetic fibers, for example, include synthetic polymeric materials and bio-plastic polymers, such as polyethylene (PE), polyethylene terephthalate (PET), polylactic acid (PLA), polyhydroxyalkanoates, polybutylene succinate. Fibers that are not synthetic, for example, include cotton, flax, hemp, rayon, and jute. Substantially free of synthetic materials means that the article or component of the article is at least about 90% or at least 95% or at least about 98% by weight void of materials where the polymerization did not happen in nature or was not naturally occurring.

### Applicator Features

The present disclosure related to absorbent articles including a tampon, and more particularly, to a tampon having an outer member and an inner member such that the inner member is slidably engaged with the outer member. The present disclosure relates to a method for forming the applicator including one of the outer member and/or inner member for use with the tampon. The outer member has an outer surface surrounding a longitudinal applicator axis. The outer member includes a barrel region and a grip region. The grip region includes greater than four circumferentially formed faces. These greater than four faces provide greater functionality and ease of use by providing multiple areas for the user to grip and securely hold the tampon during use. Each of the outer member and the inner member may include one or more layers that are spirally or convolutely wound to form the outer member and the inner member. Each of these one or more layers may comprise, consist essentially of, or consist of cellulosic material. Stated another way, each of the one or more layers may be void of synthetic fibers and films. A carboard applicator, as referred to herein, is an applicator that includes one or more layers and each of the one or more layers comprise, consists essentially of, or consists of cellulosic material and is void of synthetic fibers and films. “Synthetic” means that the polymerization did not happen in nature or was not naturally occurring. Synthetic materials are those that are chemically modified such that the polymerization was not naturally occurring. Synthetic fibers, for example, include synthetic polymeric materials and bio-plastic polymers, such as polyethylene (PE), polyethylene terephthalate (PET), polylactic acid (PLA), polyhydroxyalkanoates, polybutylene succinate. Materials that are not synthetic, for example, include cotton, flax, hemp, rayon, and jute. Free of synthetic materials means that the article or component of the article is at least about 90% or at least 95% or at least about 98% by weight void of materials where the polymerization did not happen in nature or was not naturally occurring.

Thus, the tampon of the present disclosure may have a cardboard applicator that provides greater functionality and convenience, and a more appealing ergonomic and aesthetic experience.

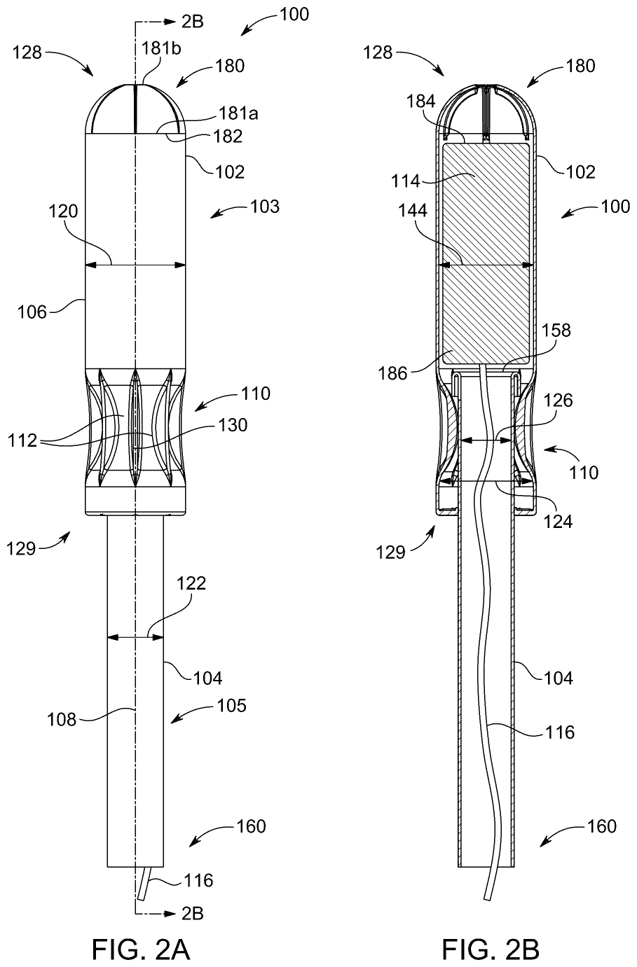

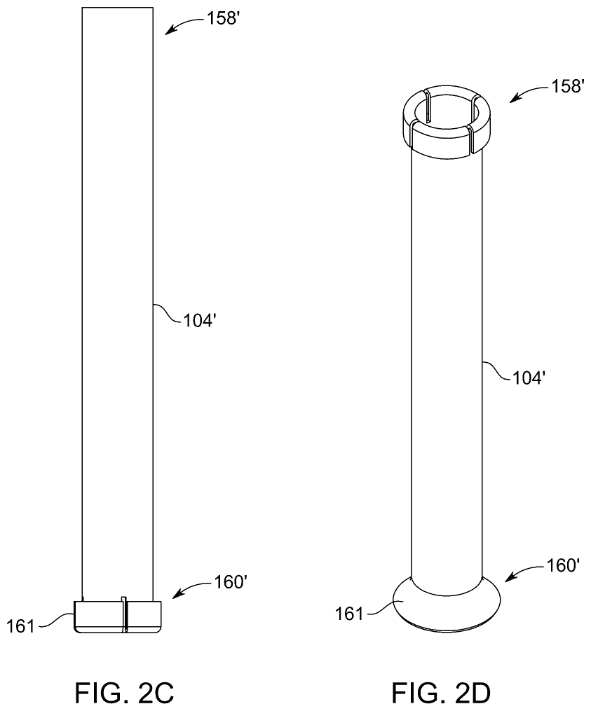

Referring to FIGS. 2A and 2B, a tampon in a self-sustaining form, including a pledget 114 having a forward end 184 and rearward end 186, and a withdrawal cord 116 attached to the pledget 114 and trailing from the rearward end 186, may be supplied inside an applicator 100. Applicator 100 may be configured to house the tampon and protect it from contamination and/or unintended deformation prior to its use.

Applicator 100 may include an outer member 103 including a hollow barrel region 102 and an inner member 105 including an ejection plunger 104 (also referred to herein as an inner member), which also may be hollow. The outer member 103, including the barrel region 102, and the ejection plunger 104 may be configured such that the ejection plunger 104 is at least partially disposed and longitudinally slidable within the barrel region 102. In some examples including the examples illustrated in FIGS. 2A and 2B, each of the barrel region 102 and the ejection plunger 104 are hollow approximately cylindrical bodies, wherein the cylindrical form of ejection plunger 104 has an external diameter 122 that is smaller than an inner diameter 144 of the barrel region 102, such that the barrel region 102 and the ejection plunger 104 may be concentrically arranged and longitudinally slidable with respect to each other in a telescoping manner. In some embodiments, the external diameter 122 of the plunger 104 or inner member 105 is less than about 70% of the external diameter 120 of the barrel region 102.

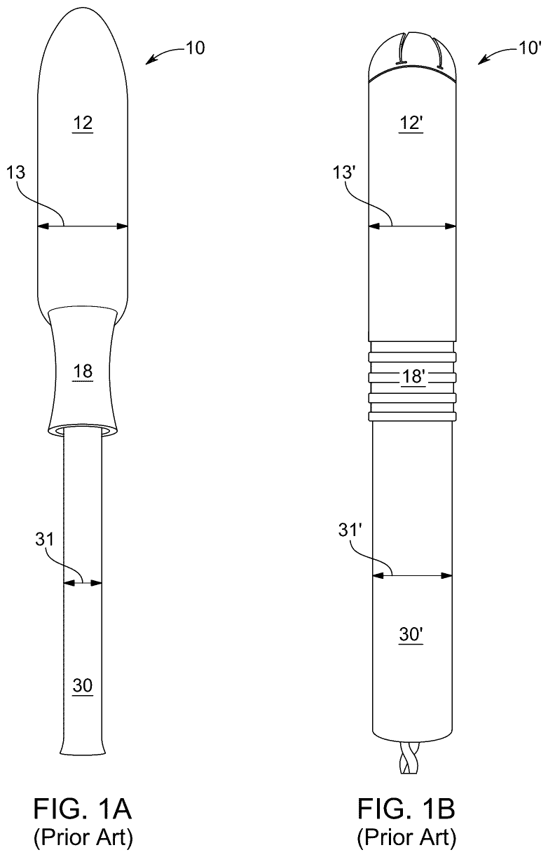

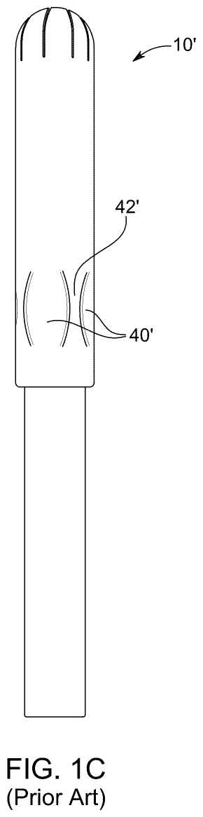

As shown in FIG. 2A, the outer member 103 includes an outer surface 106 surrounding a longitudinal applicator axis 108, the barrel region 102, and the grip region 104. In some embodiments, the barrel region 102 has an external diameter 120 that is greater than the external diameter 122 of the plunger 104. In one example embodiment, the external diameter 120 of the barrel region 102 is about 16 mm or in a range from about 12 mm to about 20 mm and the external diameter 122 of the plunger 104 is about 7 mm or in a range from about 6 mm to about 8 mm. In some embodiments, the external diameter 122 of the plunger 104 is less than about 70% or less than about 60% or less than about 50% or less than about 40% of the external diameter 120 of the barrel region 102. Unlike the conventional cardboard applicator 10′ (FIG. 1B) where the external diameter 31′ of the plunger 30′ is about 80-90% of the external diameter 13′ of the barrel region 12′, the external diameter 122 of the plunger 104 is about 50%-60% of the external diameter 120 of the barrel region 102. The inventors of the present disclosure recognized that the external diameter 31′ of the plunger 30′ in the conventional cardboard applicator 10′ had to be at least 80-90% of the external diameter 13′ of the barrel region 12′ to avoid the plunger 30′ wobbling as it is slid into or withdrawn from the barrel region 12′. The improved cardboard applicator 100 herein discloses an improved grip region 110 to accommodate the external diameter 122 of the plunger 104 only being 50-60% of the external diameter 120 of the barrel region 102 in order to prevent the undesired wobbling between the inner member 104 and outer member 102.

The outer member may be provided with additional functional features. For example, a forward end 128 of the outer member may include an insertion end region adjacent to the barrel region. The insertion end region includes a plurality of petals 180 that have proximal bases 181a having outer surfaces coincident with the general cylindrical shape of the barrel region 102, and distal ends 181b disposed radially inwardly of the proximal portions. The forward end 128 of the barrel region 102 is opposite from the grip region 110. The petals 180 and/or remainder of the barrel region 102 may be configured such that the petals 180 may flex about the proximal bases 181a such that, upon forward longitudinal pressure exerted by the forward end 184 of the pledget 114 during ejection via pressure form longitudinal forward movement of the ejection plunger 104 relative to the barrel region 102, the petals 180 will flex outwardly about their bases. This expands an opening at the forward end 128 of the barrel region 102, which allows the pledget 114 to pass forwardly through the forward end 128 of the barrel region 102, and out of the applicator 100. The petals 180 may be integrally formed with the remainder of the barrel region 102. In some examples, the petals may be formed by making cuts in a forward portion of a cylindrical precursor body of the barrel region 102, and then plastically deforming the remaining uncut portions radially inwardly, and with suitable curvature. Such deformation may be accomplished in some examples via use of a forming die or mold (not shown), and may be facilitated in some examples via application of heat and/or steam. Additionally, in some examples the barrel region 102 may be imparted with localized flexure zones 182 that demark the proximal bases 181a of the petals, which are zones about which the material of the barrel region 102 and/or petals more readily flexes or hinges, for example, as a result of a localized reduction in thickness or caliper of the material in flexure zones 182. Flexure zones 182 may be imparted by, e.g., molding or localized compression applied during the manufacturing process.

The outer member 103 may also be provided with a grip region 110 having gripping surface features (e.g., circumferential faces 112) that enable the user to tactilely identify a rearward end region 129 of the outer member 103, and also enhance gripping slip resistance during use of the applicator 100. In a simple form, such as shown in FIGS. 2A and 2B, the features of the grip region 110 may be a series of circumferential faces 112 spaced apart by respective ribs 130 about the outer surface 106 of the outer member 103 proximate its rearward end region 129. More complex gripping surface features may be provided, however, of any design desired for purposes of functionality and esthetic appeal. Gripping surface features may be imparted to barrel region 102 via the method disclosed herein that includes various steps that employ various forming techniques including molding, embossing, etc.

As illustrated in FIG. 2B, the barrel region 102 may be sized to house the tampon pledget 114 in its self-sustaining form. It may be desired that the barrel region 102 have an internal diameter 144 that is small enough relative the outer lateral dimension of the pledget 114 such that the pledget 114 cannot freely slide longitudinally within the barrel region 102 (due to slight interference or radial compression and light static friction resistance), but not so small as to create unacceptable static friction resistance and thereby require the user to exert unacceptably great force on the ejection plunger or inner member 104 to eject the pledget 114 from the outer member, when ejection is desired.

Also as suggested in FIG. 2A, it may be desired that the ejection plunger 104 have the external diameter 122 (of its cylindrical form) that is equal to or smaller than the internal diameter 144 or other inner lateral dimension of the grip region 110. It may be desired to provide such clearance within 1% of parity such that the ejection plunger 104 cannot freely and unintentionally slide coaxially/longitudinally within or out of the barrel region 102 in the absence of intentionally applied force, but such that the force required to slide the ejection plunger 104 within the grip region 110 is not so great as to be unacceptably great to the user, or even potentially destructive to the applicator.

Referring to FIGS. 2A and 2B, the ejection plunger 104 may be provided with an inner diameter that is smaller than an outer diameter or lateral dimension of the tampon pledget 114 (or other object) housed within the barrel region 102. With this size relationship, longitudinal force applied to the ejection plunger 104 urging it into the barrel region 102 will cause its forward end or first end region 158 to contact the rearward end 186 of the tampon pledget 114 (or other object) and urge the object forward and out the forward end 128 of the barrel region 102. As an alternative, or in addition to, such inner diameter size at the forward end region or first end region 158 of the ejection plunger 104 may have its circumferential edge rolled over inwardly or otherwise formed to extend radially inwardly, thereby effectively reducing the inner diameter or inner lateral dimension of the ejection plunger 104 at its forward end 158. The first end region 158 of the ejection plunger or inner member 104 may be outwardly rolled or flanged to increase the amount of area at the forward end 158 of the ejection plunger 104 that effectively contacts the rearward end 186 of the pledget 114 or other object within the barrel region 102, to transmit ejection force exerted by the user. A similar feature can be imparted to the rearward end or second end region 160 of the ejection plunger 104, to expand contact surface area and decrease focused/localized pressure on the user's finger at the circumferential rim of the rearward end 160 when the user applies ejection force at the rearward end 160, and thereby make application of ejection force more comfortable to the user's finger. The pledget 114 may be configured with an expulsion force of less than 900 gram-force (gf) or less than 800 gram-force (gf) or in a range of from about 400 to about 450 gf or from about 250 gf and about 700 gf.

In some embodiments, the barrel region 102 is manufactured such that the internal diameter 144 thereof is less than about 19 mm and preferably less than about 16 mm. Although the external diameter of typical menstrual-use tampons varies, most menstrual-use tampons currently marketed have an external diameter of less than about 19 mm. However, if one desires to use the applicator of the present disclosure for purposes other than delivery of a menstrual-use tampon to a human vaginal cavity (such as, for example, delivery of other devices, medications, etc. to other cavities in humans, or cavities in animals (veterinary use)), a barrel region 102 with a larger diameter may be desired.

The material of the outer member may be overlapped into a tubular configuration. Spirally or convolutely winding the outer member into a cylindrical tube is especially advantageous when the outer member is formed from a laminate. The reason for this is that when a laminate is circumferentially wound into a tube and a butt seam or an overlap is formed, a common problem with a rigid or stiff walled, tubular member having a relatively small diameter and a butt seam is that the seam may tend to come apart after formation if exposed to certain stress forces and/or high humidity. Accordingly, it may be preferred that the barrel region 102 be formed into a cylindrical configuration without the presence of a butt seam or an overlap.

The ejection plunger 104, also referred to herein as the inner member, may be manufactured of similar materials and in a similar layered and wound configuration as the barrel region 102, with appropriate adjustments to its size (length and outer diameter) to enable it to fit within the grip region 110 with appropriate clearance, and have sufficient length to facilitate full ejection of the tampon pledget (or other object) from the barrel region 102.

For a menstrual-use tampon, the barrel region 102 may be sized and configured to snugly house the tampon. As noted above, it may be desired that the outer member have a relatively smooth exterior surface that will facilitate comfortable insertion of the barrel region 102 into the vaginal cavity. When the exterior surface is smooth (and preferably has surface properties and/or surface treatment that provide appropriately low friction with sensitive tissue), the barrel region 102 will easily slide within the vaginal cavity without subjecting the internal tissues of the vagina to irritation or abrasion. The exterior surface of the barrel region 102 may be coated to give it appropriate low friction characteristic surface properties. Wax, polyethylene, a combination of wax and polyethylene, and cellophane are representative components of coatings that have been applied to barrel regions of tampon applicators, to facilitate comfortable use.

The applicator of the present disclosure may be used for the delivery of menstrual-use devices, such as a tampon, an intravaginal collection device (e.g., menstrual cup), and interlabial pads. The applicator of the present disclosure may be used for the delivery of a pessary. The applicator may also be useful for delivery of oral, rectal, and vaginal suppositories, as well as nasal devices, such as nasal tampons. The applicator may be used for delivery of various other materials including, medicaments, moisturizers, vitamins and minerals, spermicides, and odor controlling agents. These materials may be delivered in the form of rigid solid objects, deformable solid objects, creams, foams, gels, etc. The applicator may be adapted for human or animal/veterinary use.

FIG. 2B depicts a non-limiting example of a tampon having the pledget 114 in a self-sustaining form and having a forward end 184 and a rearward end 186 and a withdrawal cord 116 attached to the pledget 114 and extending rearward from a location proximate the rearward end 186 of the pledget 114. Generally, the “pledget” as referred to herein is that portion of the tampon that includes the main body and mass of absorbent material, but does not include the withdrawal cord 116. Tampons contemplated herein, however, are not limited to structures having the particular configuration shown in the drawing. In some embodiments, at least a portion of the cord 116 is braided. In other embodiments, there may be two different portions of the cord 116 that are each braided. In this example embodiment, one of the braided portions is a thick portion and the other of the braided portions is a thinner portion, as disclosed in U.S. Patent Publication No. 2023/0058874 that is incorporated by reference herein.

As shown in FIG. 2B, the pledget 114 of the tampon has the forward end 184 and the rearward end 186. During manufacture of the tampons the pledget 114 may be folded, bunched, compressed and/or otherwise formed in size and shape, from its initially manufactured configuration into a generally cylindrical and/or capsule-shaped configuration (e.g., as shown in FIG. 2B) along a radial direction, the lateral direction, longitudinal direction, or in some combination thereof.

FIG. 2C is a longitudinal side view of an inner member 104′ of the tampon product of FIG. 2A with a flange 161 at a second end region 160′ thereof. During use of the applicator 100, the plunger 104′ includes the forward end 158′ and the rearward end 160′ opposite to the forward end 158′. The forward end 158′ of the plunger 104′ is configured to engage a portion of the pledget 114 and the rearward end 160′ includes the flange 161. In some embodiments, the flange 161 can have a diameter greater than the internal diameter of the barrel region 102 at the rearward end region 129 or the flange can have a diameter greater than a portion of the internal diameter of the grip region 110, to keep the plunger 104′ from unintentionally going through the barrel region 102 and out the forward end of the outer member, and to provide a surface on which the consumer or user can push to expel the pledget 114 from the forward end 128 of the barrel region 102, such as shown in FIG. 2D. In some embodiments, the flange 161 may be a pushed-out petal, a compressed portion, an embossed portion, or may include added material to increase the diameter of the plunger 104′ at the rearward end 160′. It is also to be appreciated that each of the first end region 158′ and/or the second end region 160′ may include a portion that has been flanged or rolled inwardly or outwardly.

### Machine Used to Form the Applicator

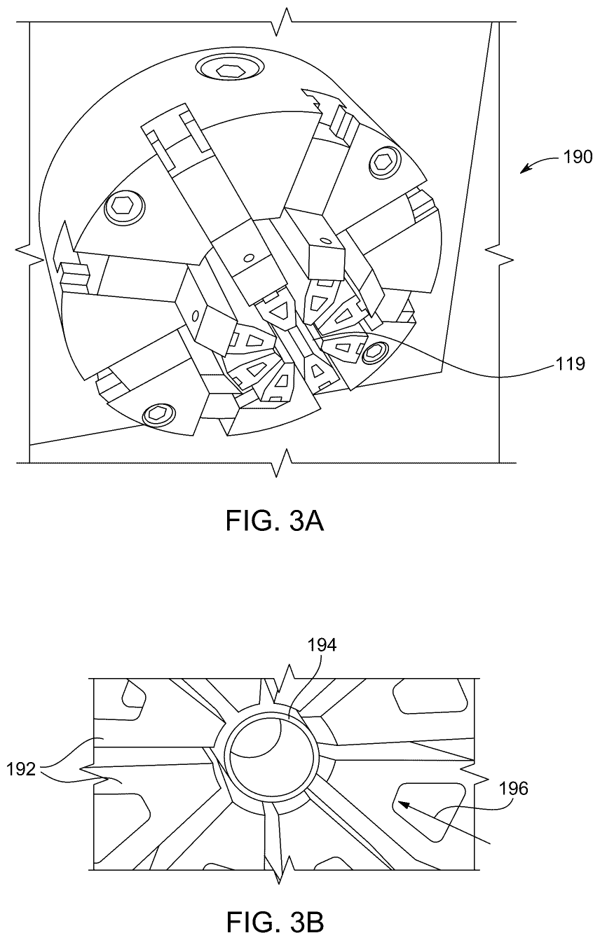

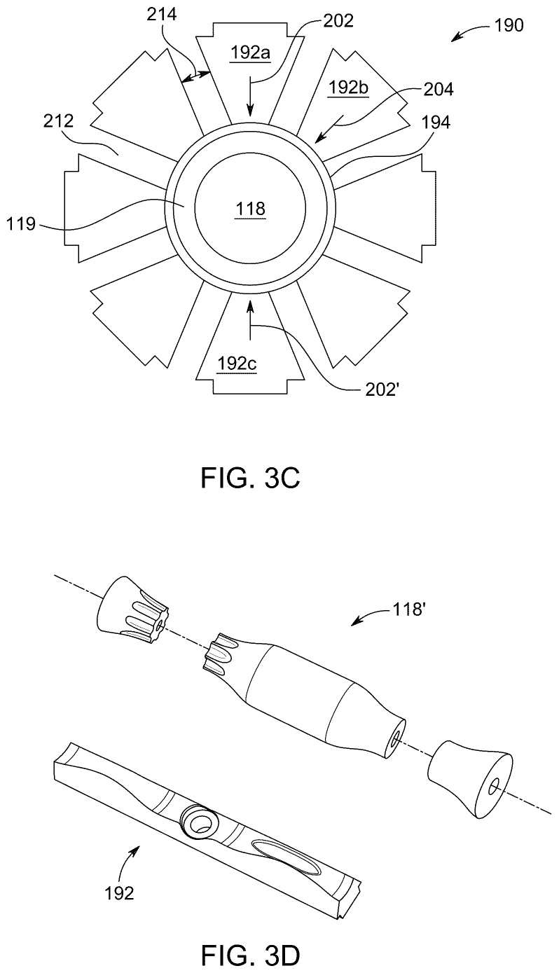

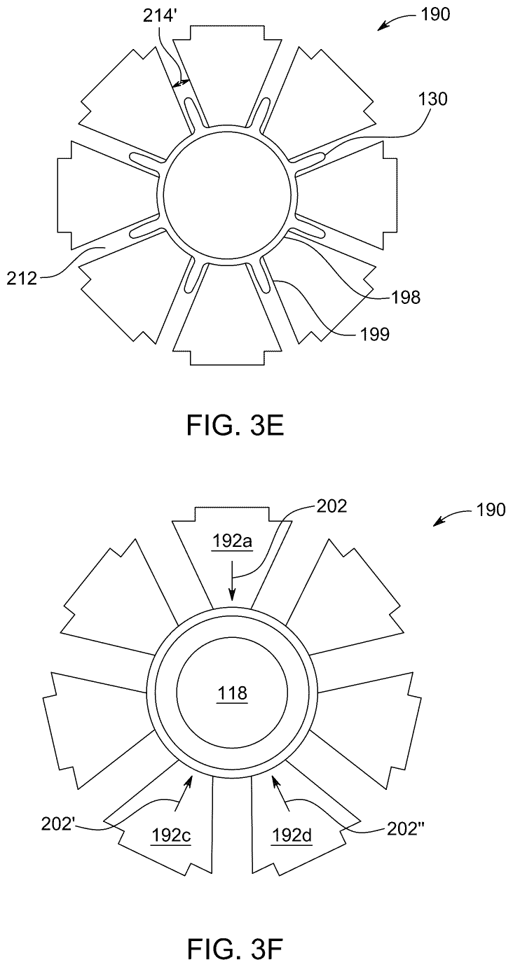

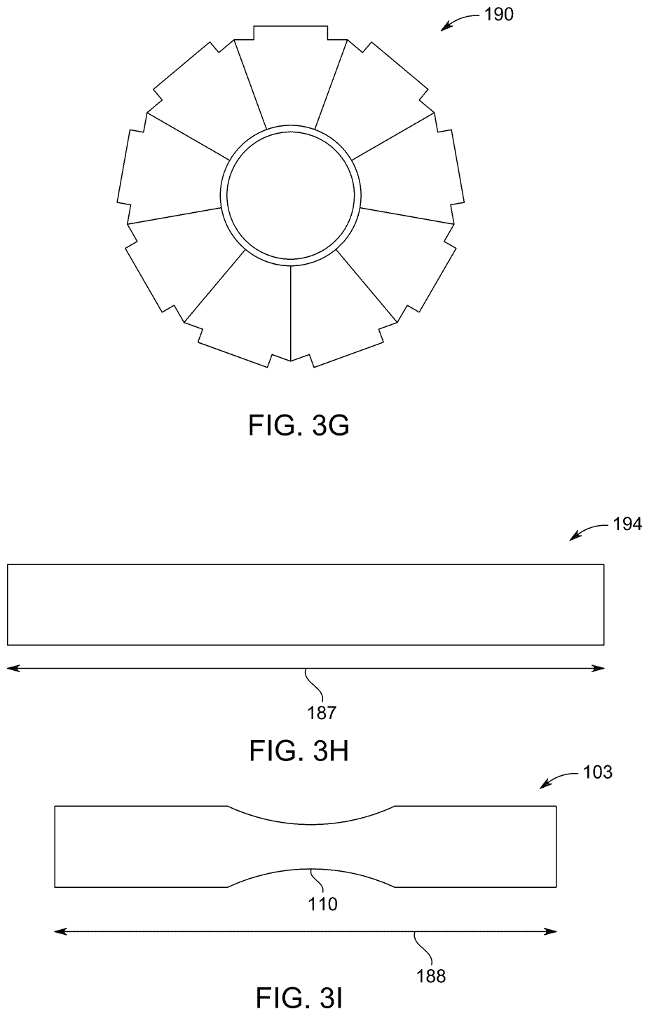

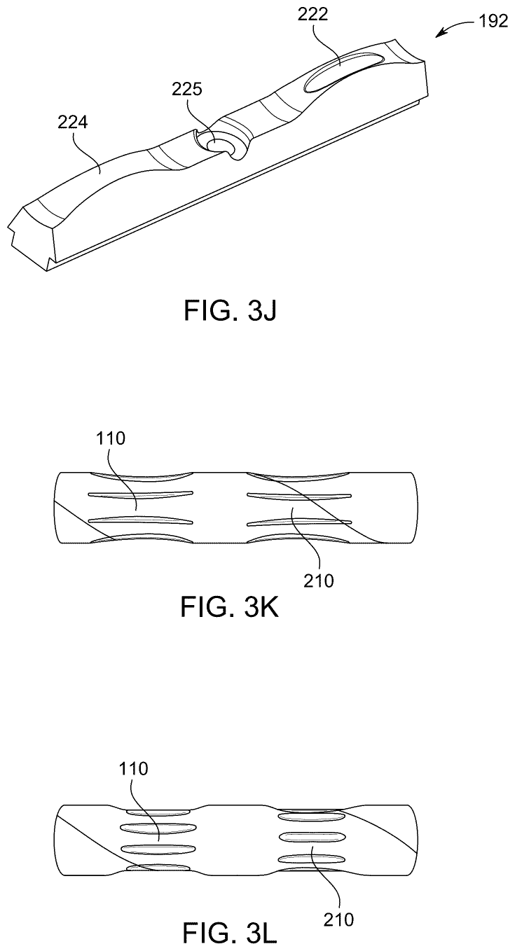

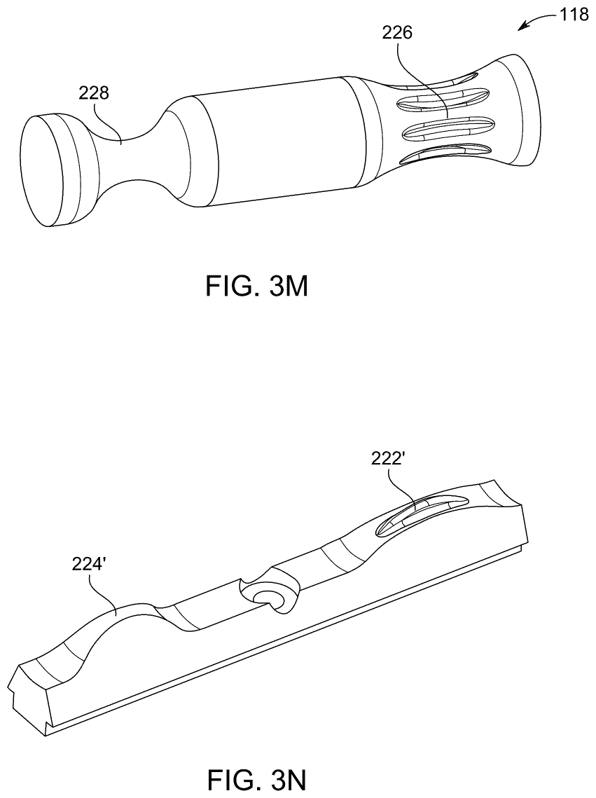

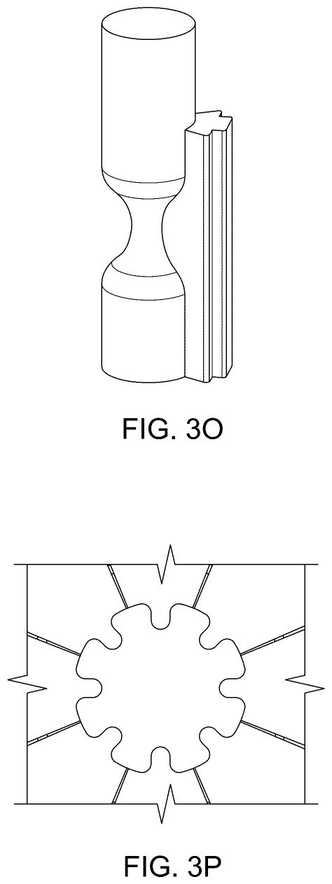

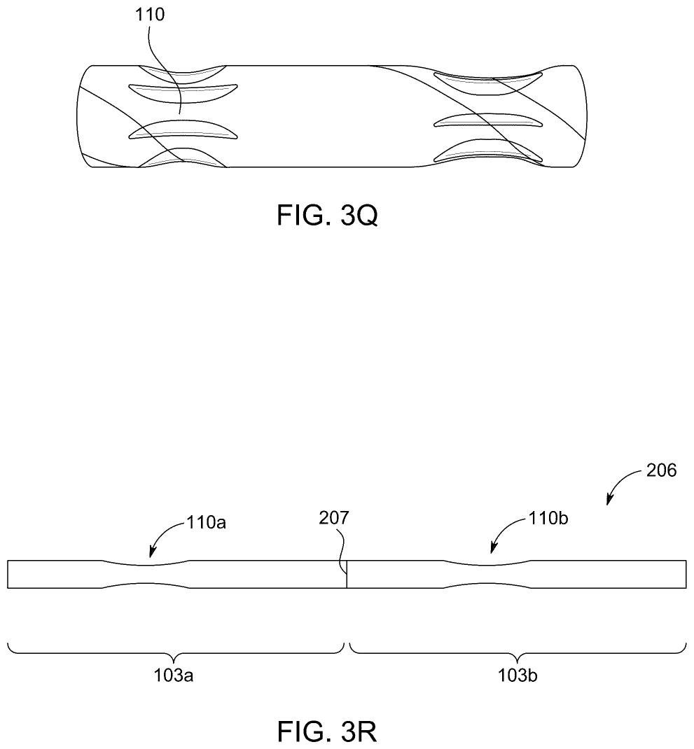

A machine that can be used to form the tampon applicator disclosed herein is now discussed. FIG. 3A is a perspective view of a compression device 190 used to form circumferential faces 112 in the grip region 110 of the applicator 100 of FIG. 2A. FIG. 3B is a perspective view of a plurality of chucks or forming members 192 of the machine 190 of FIG. 3A that move radially inward to form the circumferential faces 112 in the grip region 110 of the applicator 100. As shown in FIG. 3B to form the grip region 110 in the applicator 100, a tube 194 is provided, which may be a cardboard or paper tube, and is positioned within an opening of the compression device 190. In some embodiments, the tube 194 is similar to the outer member 103 of FIG. 2A without the grip region 110 and/or petals 180. In other embodiments, the tube 194 is similar to the inner member 105 of FIG. 2A.

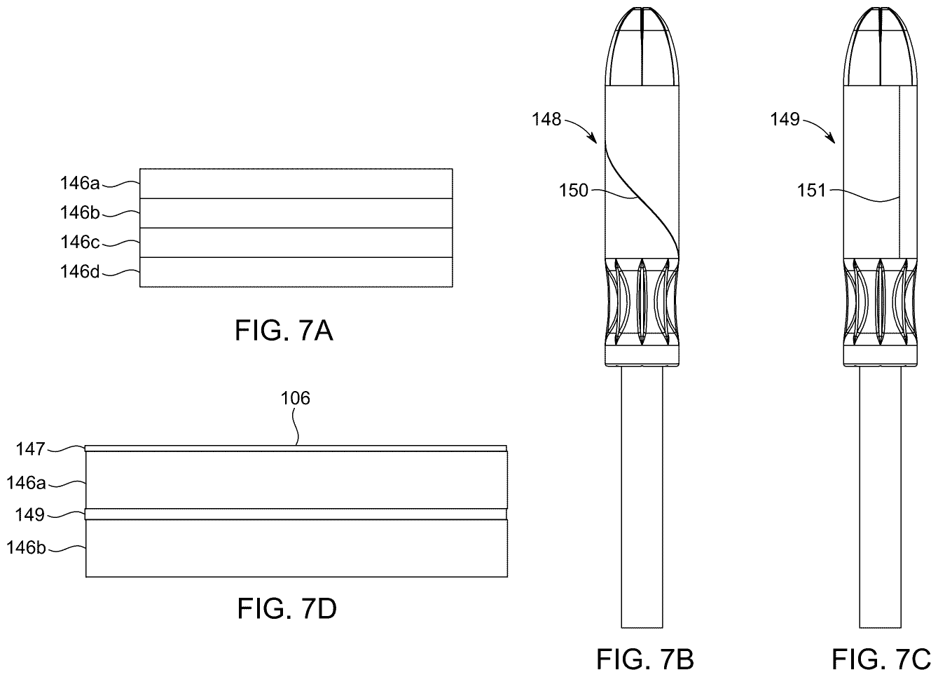

In some embodiments, the tube 194 is a spirally wound group of one or more layers, at least one of which includes cellulosic material (e.g., spiral wound layers 148 with a non-linear seam 150 as discussed with respect to FIG. 7B). In other embodiments, the tube 194 is convolutely wound layers of material including at least one layer of cellulosic material (e.g., convolutely wound layers 149 with a linear seam 151 as discussed with respect to FIG. 7C). In these embodiments the tube 194 is formed by winding one or more layers (e.g., layers 146a through 146d in FIG. 7A and layers 146a and 146b in FIG. 7D) in one or more revolutions.

In some embodiments, the tube 194 is formed by pulp molding. It has been found that cellulose pulp-based composites may be formulated, that are suitable for injection molding in conventional molds adapted for use to mold applicator assembly components and other molded objects from polymer resins (plastics). The pulp-based composites may be injection molded to form such objects, having sizes, shapes, surface finishes, configurations and relatively fine/small feature details, comparable to similar objects molded of plastics. Moreover, such composites may be formulated without inclusion of components derived from petroleum (such as plastics with petroleum or petroleum-derived components or precursor materials); such that the molded products are relatively rapidly dispersible when immersed in water; and such that they are biodegradable, which may contribute to making them less of a burden on water treatment and/or waste management resources. Preferably, one, more, or all components of an applicator assembly as described herein will contain less than 10 percent by weight, more preferably less than 5 percent by weight, and even more preferably substantially no, petroleum, petroleum derivatives, and/or plastics with petroleum or petroleum-derived components or precursor materials.

Further, materials manufactured from or containing cellulose pulp such as wood pulp may be recyclable. Applicator assemblies and components thereof as described herein may be manufactured from molding composites formulated to render the resulting molded parts recyclable in conventional paper recycling operations. Additional details regarding the pulp molding process used to form the tube 194 is disclosed in U.S. Published Application Number 2022-0296434 A1, which is incorporated by reference herein. Since the tube 194 can be used to form the outer member 103 or the inner member 105 of the applicator 100, the above discussed pulp molding technique can be used to form either the outer member 103 or inner member 105 of the applicator 100.

In use, after positioning the tube 194 in the opening defined by the compression device 190, the forming members 192 are moved in an inward radial direction 196 and engage the outer surface of the tube 194 to form the circumferential faces 112 and ribs 130 therebetween. After forming the circumferential faces 112, the forming members 192 are moved in an opposite (outward) radial direction. The tube 194 is then moved or advanced such that a different section of the tube 194 is positioned within the opening and the process is repeated so that the grip region 110 including the circumferential faces 112 and ribs 130 therebetween are formed at a plurality of spaced apart regions along the tube 194. The tube 194 is then removed from the compression device 190 and cut into individual parts where each part is the outer member 103 with the formed barrel region 102 of the applicator 100.

In other embodiments, the tube 194 may be first cut into a plurality of parts and each respective part is positioned in the opening of the compression device 190 and the forming members 192 are used to form the circumferential faces 112 in each part of the tube 194, resulting in the outer member 103 with the grip region 110. The forming members 192 may be used to form the circumferential faces 112 without using a mandrel, a spindle, or any supporting material or device positioned inside the tube 194. The inventors of the present invention surprisingly found that providing a supporting material or device within the tube 194 was not required in order to radially compress the tube 194 to form the circumferential faces 112 and ribs 130 of the grip region 110.

Although the machine 190 of FIGS. 3A and 3B is a radial compression machine that compresses the tube 194 in the inner radial direction 196, the embodiments of the present invention is not limited to such a radial compression device. In other embodiments, a compression device can be utilized to form one or more components of the applicator 100 by compressing the tube 194 in a direction other than the radial direction. In one example embodiment, a compression device is utilized to compress one or more ends of the tube 194 in a longitudinal direction (e.g., parallel to the longitudinal applicator axis 108).

The inventors of the present invention recognized that the compression device 190 should be operated such that the paper or cardboard tube 194 is compressed to form the circumferential faces 112 without forming slits or holes in the tube 194. The inventors of the present disclosure surprisingly found that the tube 194 material may be compressed to form the circumferential faces 112 without affecting the color of the tube 194. As shown in FIG. 3E, in some embodiments, the forming members 192 of the compression device 190 radially compress the tube 194 material and pinch the material between two adjacent forming members 192 to form the circumferential faces 112 with ribs 130 therebetween. FIG. 3E depicts that the forming members 192 pinch the tube 194 material in the space 212 between adjacent forming members 192 which forms the ribs 130. In some embodiments, heat is used in conjunction with the compression and squeezing of the forming members 192 but in other embodiments no heat is employed.

The inventors of the present disclosure noted that there is some rebound to the cardboard tube 194 material as it is compressed to form the circumferential faces 112 and ribs 130. The inventors of the present invention further recognized that the extent of the rebound depends on various factors including the structure of the layers of the tube 194. In one example embodiment, the inventors recognized that if the glue or adhesive between adjacent layers of the tube 194 has not yet cured, the extent of the rebound is reduced since the curing of the adhesive assists in stiffening the cellulosic material of the tube 194 so that the material is held in the compressed position (e.g., ribs 130 with a particular height). Thus, in some embodiments, the inventors recognized that in order to reduce the extent of the rebound of the material, the tube 194 is compressed with the machine 190 before the adhesive has cured.

The inventors of the present disclosure noted that for the conventional cardboard applicator 10′ (FIG. 1C), the greatest number of circumferential faces formed in the cardboard material is four. Thus, the inventors of the present disclosure herein configured the machine 190 to form greater than four circumferential faces 112 in the grip region 110. This is due to a larger number of circumferential faces in the grip region enhancing the control of the user over the barrel region 102 and/or the plunger 104. Unlike plastic material, which is relatively easy to mold and shape, the inventors of the present disclosure recognized that paper material is more challenging to mold and shape and thus had to overcome design obstacles not present in forming plastic applicators. For example, paper or carboard is prone to tearing when too much pressure is applied, and paper or cardboard tends to fold and produce wrinkles that would likely be aesthetically displeasing to consumers.

As previously discussed, conventional cardboard applicators 10′ (FIG. 1B) feature the barrel region 12′ with the external barrel diameter 13′ that is relatively close to the external plunger diameter 31′ of the plunger 30′. The inventors of the present disclosure expected that the compression and pinching of the paper or cardboard tube 194 with the forming members 192 would cause undesirable fold lines in the tube 194. Instead, the inventors of the present disclosure surprisingly found that the tube 194 tolerates the compression and squeezing of the forming members 192 relatively well, resulting in a surprisingly smooth surface to the circumferential faces 112 and ribs 130. Additionally, the inventors of the present disclosure expected that the compression and pinching of the tube 194 by the forming members 192 would require an additional heating step to form the circumferential faces 112 and ribs 130. However, the inventors surprisingly found that no additional heating step was required.

FIG. 3C is an end view of a stabilizing rod positioned within an interior portion of the applicator prior to the forming members forming the circumferential faces. Although FIGS. 3A and 3B disclose an embodiment where no mandrel or supporting member is positioned within the tube 194 as it is compressed by the forming members 192, in other embodiments a mandrel or supporting member can be used. FIG. 3C depicts an embodiment where a stabilizing rod or mandrel 118 is positioned within the interior portion 119 of the tube 194 prior to compression of the tube 194 with the forming members 192. However, as previously disclosed, the inventors of the present invention surprisingly found that the stabilizing rod or mandrel 118 need not be positioned within the tube 194 in order to radially compress the tube 194 to form the grip region 110.

As shown in FIG. 3C, in this embodiment an even number (e.g., eight) of forming members 192 are provided in the machine 190. The forming members 192 have an initial spacing 214 in a circumferential direction when initially compressing the tube 194. As the forming members 192 continue to radially compress the tube 194, the forming members 192 have a reduced spacing 214′ (FIG. 3E) in the circumferential direction when the forming members 192 are in a fully compressed position (e.g., when they reach an inner radial limit of movement).

As further shown in FIG. 3C, the even number of forming members 192 apply inward radial pressure so that radially opposite forming members 192a, 192c apply radial pressure 202, 202′ that is substantially equal in magnitude and opposite in direction. Thus, as shown in FIG. 3C the first forming member 192a applies a first radial pressure 202 in a first direction (e.g., downward direction in FIG. 3C) whereas the second forming member 192c that is radially opposite from the first forming member 192a applies a second radial pressure 202′ that is substantially equal in magnitude but opposite in direction (e.g., upward direction in FIG. 3C) to the first radial pressure 202. In this embodiment, each forming member 192 of the machine 190 applies radial pressure that is substantially equal in magnitude but opposite in direction from a corresponding radially opposite forming member 192. The inventors of the present invention recognized that for a machine 190 employing an even number of forming members 192, this condition of radially opposite forming members applying pressure that is substantially equal in magnitude but opposite in direction advantageously permits the grip region 110 to be formed within having to employ the mandrel or stabilizing rod 118 within the tube 194. As further shown in FIG. 3C, an inward radial pressure 204 may be applied by a forming member 192b that is adjacent to the forming member 192a may be equal in magnitude or different in magnitude than the inward radial pressure 202 applied by the forming member 192a. Stated another way, a first forming member 192a may be positioned adjacent or next to a second forming member 192b, and the first forming member 192a may be configured to apply a first forming pressure and the second forming member may be configured to apply a second forming pressure. The first forming pressure may be the same as or different than the first forming pressure. Although FIG. 3C depicts eight forming members 192, in other embodiments less or more than eight forming members 192 can be used.

The stabilizing rod or mandrel 118 positioned within the interior portion 119 of the tube 194 can be one-piece or feature a plurality of interconnected pieces. FIG. 3D is a perspective view of a forming member 192 of the machine 190 and a stabilizing rod 118′ comprising a plurality of interconnected members. Thus, in this embodiment the stabilizing rod or mandrel 118′ can be positioned within the interior portion 119 of the tube 194 by first positioning one of the pieces (e.g., long piece in FIG. 3D) within the interior portion 119 and subsequently attaching one of the end pieces to opposite ends of the long piece within the interior portion 119. The inventors of the present invention recognized that one advantage of the stabilizing rod or mandrel 118′ featuring multiple pieces is that the stabilizing rod or mandrel 118′ can be more easily removed from the interior portion 119 of the tube 194 after compression of the tube 194. This is because the end pieces can be first detached from the long piece of the mandrel 118′ after which the long piece of the mandrel 118′ can be subsequently removed.

FIG. 3E is an end view of the forming members of FIG. 3B radially compressing the applicator and pinching the tube 194 between the forming members to form the ribs 130. As shown in FIG. 3E, in some embodiments each forming member 192 has a face impression region 198 that is configured and/or shaped to form the circumferential face 112 in the grip region 110 and a rib impression region 199 that is configured and/or shaped to form the rib 130 as the forming members 192 are compressed from the first radial position (with first circumferential spacing 214 of FIG. 3C) to the second radial position (with second reduced circumferential spacing 214′ of FIG. 3E). As shown in FIG. 3E, in some embodiments each of the eight ribs 130 (e.g., greater than four) are formed by compressing a portion of the tube 194 between adjacent forming members 192.

As shown in FIGS. 3C and 3E, as the forming members 192 move radially inward from the first radial position (with first circumferential spacing 214 of FIG. 3C) to the second radial position (with second reduced circumferential spacing 214′ of FIG. 3E) the forming members 192 simultaneously apply pressure to the tube 194 so to form the grip region 110. As previously discussed, with the even number of forming members 192 the applied pressure of the forming members 192 meets certain criteria (e.g., radially opposite forming members 192 apply pressure that is substantially equal in magnitude and opposite in direction).

Although FIGS. 3A through 3E discussed an embodiment of the machine 190 employing an even number of forming members 192, in other embodiments the machine 190 employs an odd number of forming members 192 to compress the tube 194. FIG. 3F is an end view of an odd number of forming members 192 of a machine 190 that move radially inward to form the circumferential faces 112 in the grip region 110 of the applicator. As shown in FIG. 3F, in some embodiments, each forming member 192 of the machine 190 applies an inward radial pressure 202, 202′, 202″ that is substantially equal in magnitude but different in direction on the tube 194. Also, in one example embodiment as shown in FIG. 3F, each forming member 192 of the machine 190 does not feature a corresponding forming member 192 that is positioned radially opposite of the forming member 192, as with the even number arrangement of forming members 192 in FIG. 3E. Thus, in the embodiment of the odd number of forming members 192 in FIG. 3F, each forming member 192 does not apply an inward radial pressure that is equal in magnitude and opposite in direction to an inward radial pressure applied by a radially opposite forming member 192. The odd number of forming members 192 in FIG. 3F may be arranged around the machine 190 with equal circumferential spacing and each of the forming members 192 may apply a substantially equal radial pressure.

Although the embodiment of FIGS. 3A through 3E features the forming members 192 being circumferentially spaced by the initial spacing 214 (FIG. 3C) and the subsequent reduced spacing 214′ (FIG. 3E) during radial compression of the tube 194, in other embodiments the machine 190 and forming members 192 are arranged such that no circumferential spacing is provided between the forming members 192. FIG. 3G is an end view of the forming members 192 of FIG. 3B radially compressing the tube 194 with no space between adjacent forming members 192. In one example embodiment, the forming members 192 move radially inward from having an initial circumferential spacing 214 in FIG. 3C to having no circumferential spacing in FIG. 3G as the forming members 192 radially compress the tube 194 to form the grip region 110. In other embodiments, no circumferential spacing is provided between the forming members 192 throughout the radial compression of the tube 194 to form the grip region 110.

In one example embodiment, since there is no circumferential spacing between the forming members 192 the ribs 130 are not formed in the grip region 110 and thus a concave profile is formed in the grip region 110 along the longitudinal applicator axis 108 (FIG. 3I). The inventors recognized that with the embodiments of FIGS. 3A through 3E, upon radially compressing the tube 194 inwards the ribs 130 were formed between the adjacent forming members 192 as a result of the circumferential spacing 214, 214′ therebetween throughout the compression. However, since the embodiment of FIG. 3G features no circumferential spacing between the forming members 192, the ribs 130 are not formed and thus the shape of the compressed grip region would vary from the grip region formed using the forming members of FIGS. 3A through 3F. In one example embodiment, a length of the tube 194 would vary due to the radial compression of the tube 194. FIGS. 3H and 3I are side views of the tube 194 respectively before and after the compression by the forming members 192 in FIG. 3G. Thus, in this example embodiment the tube 194 has a first length 187 prior to compression by the forming members 192 forming the grip region 110. After compression by the forming members 192 and formation of the grip region 110, the tube 194 has a second length 188 that is less than the first length 187.

Although the forming members 192 of FIGS. 3A through 3G feature a circumferential surface, this is just one example profile of the forming members 192. In other embodiments, the forming members 192 can have any profile, depending on the desired characteristics of the grip region 110. FIG. 3J is a top perspective view of a forming member 192 secured to the machine 190 of FIG. 3A having different profiles at different respective ends thereof. As shown in FIG. 3J, the forming member 192 defines a fastener opening 225 through which a fastener is passed to secure the forming member 192 to the frame of the machine 190. On a first side of the forming member 192, a first profile 222 is provided having a convex shape and including a concave portion within the convex shape. On an opposite second side of the forming member 192, a second profile 224 is provided that features a convex portion. To use the profile 222 or 224 of the forming member 192, the forming member 192 is oriented and secured to the machine 190 so that the desired profile 222, 224 is positioned to radially compress the tube 194 when the machine 190 is activated. FIG. 3K is a side view of a plurality of grip regions 110, 210 formed in the tube 194 using the second profile 224 on the second end of the forming member 192 of FIG. 3J. FIG. 3L is a side view of a plurality of grip regions 110, 210 formed in the tube 194 using the first profile 222 on the first end of the forming member 192 of FIG. 3J. In the example embodiment of FIGS. 3K and 3L where multiple grip regions 110, 210 are formed in the tube 194, the forming members 192 are used to radially compress both grip regions 110, 210 in the tube 194. In one example embodiment, after the forming members 192 radially compress the first grip region 110, the tube 194 is moved in a longitudinal direction and the forming members 192 subsequently radially compress the second grip region 210.

Although FIG. 3C depicts the stabilizing rod or mandrel 118 having a cylindrical profile, in other embodiments, the stabilizing rod or mandrel positioned within the interior portion 119 of the tube 194 can have a different profile, such as a non-cylindrical profile. In some embodiments, the profile of the stabilizing rod or mandrel can have a complimentary profile with the profile of the forming member 192. The inventors of the present invention recognized that this complimentary profile relationship between the stabilizing rod or mandrel and the forming member may enhance the formation of various features of the grip region. Thus, in some example embodiments, where the forming member 192 has a convex profile, the stabilizing rod or mandrel has a concave profile (e.g., having an equal in magnitude but opposite in sign radius of curvature). FIGS. 3M through 3P are various views showing a stabilizing rod 118 and a forming member 192 used in the machine 190 of FIG. 3A which have complimentary profiles. As shown in FIG. 3M the stabilizing rod or mandrel 118′ has a first profile 226 (e.g., circumferentially spaced convex portions) at a first end and a second profile 228 (e.g., concave portion) at a second end. FIG. 3N depicts a forming member 192 featuring a first profile 222′ (concave portion) at a first end and a second profile 224′ (convex portion) at a second end. FIG. 3O depicts the complimentary intermeshing of the second profile 228 of the stabilizing rod 118′ with the second profile 224′ of the forming member 192. FIG. 3P depicts the intermeshing of the plurality of circumferentially arranged forming members 192 with the stabilizing rod 118′. FIG. 3Q is a side view of a plurality of grip regions 110, 210 formed using the second profile 224′ of the forming member 192 and second profile 228 of the stabilizing rod 118′ of FIGS. 3M through 3P.

The various profiles of the stabilizing rod and forming members shown in FIGS. 3A through 3P are merely some example profiles of the stabilizing rod and forming members that can be used. In other embodiments other profiles of the stabilizing rod and forming member can be employed, depending on the desired shape and profile of the grip region. Additionally, as previously discussed, the stabilizing rod is optional and thus in some embodiments the profile of the forming member is selected to form a grip region having desired shape characteristics without use or consideration of a stabilizing rod profile. As illustrated in FIG. 3N, the forming members may include more than one profile 222′ and 224′. A forming member having more than one profile may include the same profile or different profiles, such as illustrated in FIG. 3N. The forming member with multiple profiles may be used to from two grip regions on a tube, and the tube may be subsequently cut or separated between the two grip regions to from two separate applicators that each have a grip region. Forming multiple grip regions on the tube may aid in increasing processing speeds and may also allow for different grips to be formed on different applicators to further customize user experience.

In some embodiments, the tube 194 of FIGS. 3A and 3B is equivalent to the outer member 103 without the grip region 110. Thus, the tube may have a length that is about equal to a desired length of the outer member 103. Thus, after compression of the tube 194 with the forming members 192, the outer member 103 with the grip region 110 is formed having the desired length. However, in other embodiments the tube 194 is an applicator parent roll which has a length that is much greater than the desired length of the outer member 103. In these embodiments, the tube 194 is compressed at multiple regions at the same time or over time so to form multiple grip regions 110 along the tube 194 after which the tube 194 is cut at incremental lengths that are about equal to a desired length of the outer member 103. FIG. 3R is an applicator parent roll 206 with a plurality of spaced apart grip regions 110 simultaneously formed by a respective plurality of machines 190 each having the forming members 192. In one example embodiment, the multiple spaced apart grip regions 110 are simultaneously formed by multiple spaced apart machines 190 having a respective set of forming members 192 which radially compress the tube 194 at the incremental lengths so to form the multiple spaced apart grip regions 110. In other example embodiments, the multiple spaced apart grip regions 110 in the applicator parent roll 206 are formed by the same machine 190, where after each grip region 110 is formed the tube 194 is moved in the longitudinal direction by the incremental length and then the machine 190 compresses the next grip region 110 until the multiple grip regions 110 are formed along the applicator parent roll 206. After each of the multiple spaced apart grip regions 110 are formed in the applicator parent roll 206, the applicator parent roll 206 is cut along a cut line 207 depicted in FIG. 3R. It should be noted that FIG. 3R depicts two spaced apart grip regions 110a, 110b formed in two incremental lengths along the applicator parent roll 206. However, this is merely for ease of illustration and in other embodiments more than two spaced apart grip regions 110 can be formed over more than two incremental lengths of the applicator parent roll 206 resulting in more than two outer members 103 after each incremental length is cut along the cut line 207.

### Characteristics of the Formed Applicator

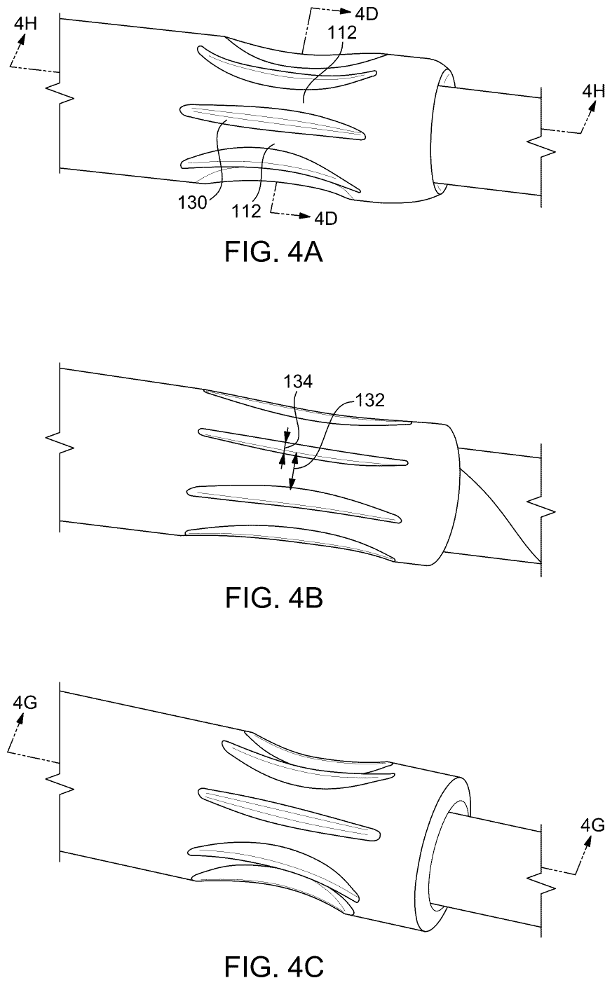

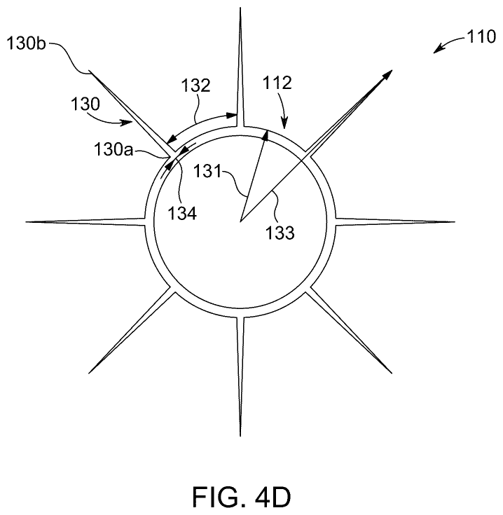

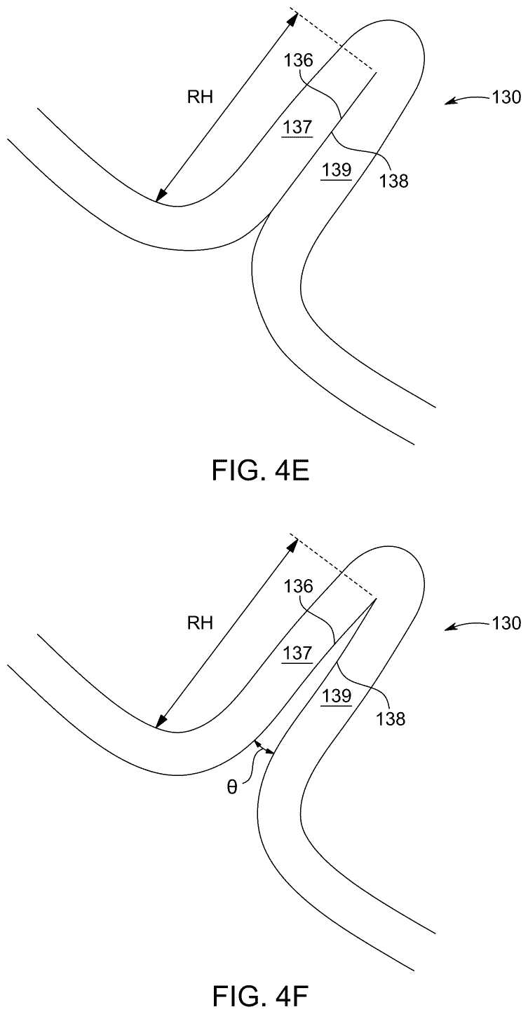

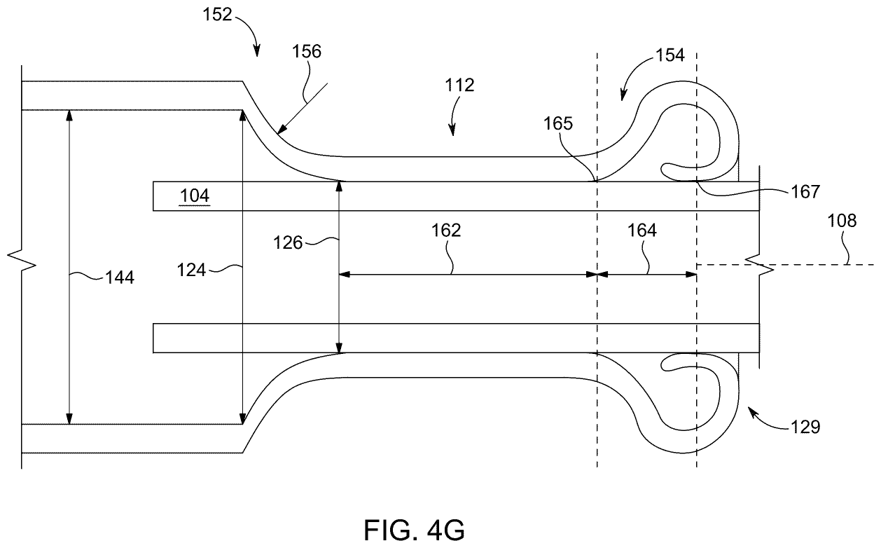

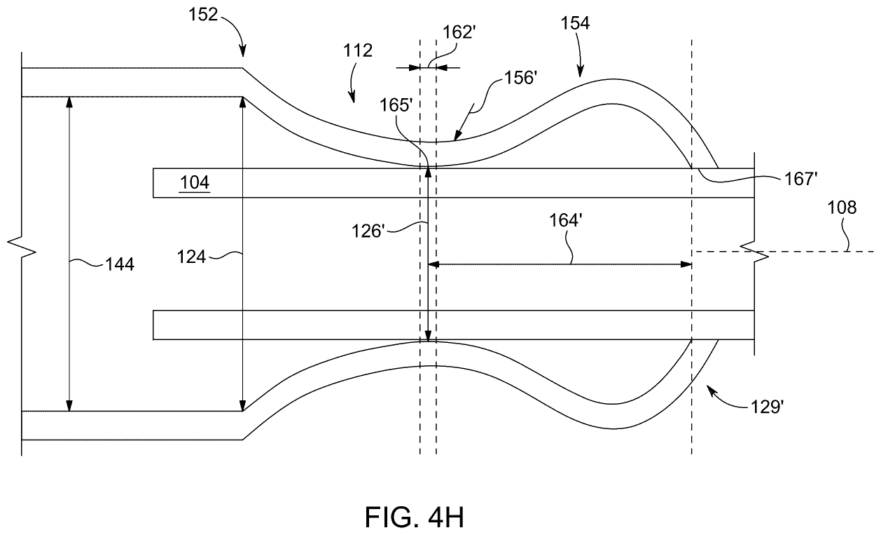

FIGS. 4A through 4C are perspective and top views of a plurality of circumferential faces 112 and ribs 130 therebetween of the grip region 110 of the applicator of FIG. 2A. In an embodiment, the circumferential faces 112 and ribs 130 therebetween are formed using any of the forming members 192 and compression device 190 as discussed herein, such as illustrated in FIGS. 3A through 3O. The circumferential faces may extend from an upper grip region to a lower grip region, as shown in FIGS. 4A-4C. Each of the faces 112 of the grip region 110, at least over a portion of the grip region, may be at least one of concave and convex. For example, two or more of the faces 112 may include a first concave region, a substantially planar region, and a second concave region. In another example, two or more of the faces 112 may be at least one of concave and convex extending from the upper grip region to the lower grip region. Each of the faces 112 may be identical in terms of shape and/or dimension. Each face 112 has one or more of a same width (measured perpendicular to the longitudinal applicator axis 108), a same length (measured along the longitudinal applicator axis 108) and a same shape (e.g., same concave or convex structure). In these embodiments, when operating the compression device 190 with the forming members 192 of FIGS. 3A through 3O, the compression by the forming members 192 is symmetrical in the inner radial direction 196 and/or each forming members 192 has a same shape or curvature to thereby compress a same-shaped circumferential face 112. In some embodiments, as shown in FIG. 3F, if an odd number of circumferential faces 112 are to be formed in the paper tube 194 then the compression force of the forming members 192 are to be symmetrical (e.g., the forming members 192 of FIG. 3F are symmetrically arranged around the tube 194 in the circumferential direction and the applied pressure 202, 202′, 202″ from each forming members 192 is equal in magnitude). In some embodiments, as shown in FIGS. 3C and 3E, if an even number of circumferential faces 112 are to be formed in the cardboard tube 194 then forming members 192a, 192c which are radially opposite from each other have the same shape (e.g., be compressed by forming members 192 having a same shape) and apply pressure 202, 202′ that is substantially equal in magnitude and opposite in direction to each other. However, in other embodiments, the forming members 192 of the machine 190 used to compress the tube 194 need not have the same profile or design. This arrangement is utilized when the desired profile of the grip region 110 includes circumferential faces 112 with different shapes or profiles around the circumference of the grip region 110. In some embodiments, the different circumferential faces 112 are formed with different shapes (e.g., different depths of engagement).

As further shown in FIGS. 4A through 4C, in some embodiments the grip region 110 includes greater than four circumferentially formed faces 112. In other embodiments, the grip region 110 includes greater than or equal to six circumferential faces 112. In still other embodiments, the grip region 110 includes greater than or equal to eight circumferentially formed faces 112. As further shown in FIGS. 4A through 4C, the grip region 110 is continuously circumferential so that there are no slits or openings in the grip region 110. As further shown in FIGS. 4A through 4C, in some embodiments the grip region 110 comprises greater than four outwardly projecting ribs 130, where each rib 130 is positioned between two adjacent circumferential faces 112. However, as discussed with respect to FIGS. 3G through 3I, in some embodiments where the forming members 192 are not circumferentially spaced when compressing the tube 194 or no ribs 130 are formed and instead the grip region 110 has a concave shape in the longitudinal direction without individual circumferential faces 112 (FIG. 3I). The inventors of the present disclosure recognized that an increased number of circumferential faces enhance the user ability to grip the grip region 110. The peak region of the rib may be substantially planar with the external surface of the barrel region, or the peak region of the rib may be concave and/or convex with respect to the external surface of the barrel region. If the peak region of the rib is convex, the convexity of the peak region of the rib is less than the convexity of the face. Stated another way, the radius of curvature of the peak region of the rib is different and/or greater than the radius of curvature of the face.

In some embodiments, the circumferential faces 112 of the grip region 110 include one or more indicia, such as printed indicia and/or tactile indicia. In one example embodiment, such tactile indicia feature embossed designs that may be formed through the same process used to form the circumferential faces using the compression device or in a subsequent or proceeding separate process. In still other example embodiments, additional material may be included outside of the grip region 110. Such additional material may include printed material (e.g., graphics, alphanumeric characters, etc.) or a material additive to create texture (tactile indicia) in the grip region 110. In some embodiments, a first face comprises a first indicia and a second face comprises a second indicia. The first indica and the second indica may be the same or different. Further, the first indicia and the second indicia may be printed indicia and/or tactile indica.

FIG. 4D is a cross-sectional view of the grip region 110 of FIG. 4A taken along the line 4D-4D. As shown in FIGS. 4B and 4D, in some embodiments, each of the circumferential faces 112 has a face circumferential width 132 and each of the ribs 130 has a rib circumferential width 134. In some embodiments, the face circumferential width 132 is greater than the rib circumferential width 134. In some embodiments, each of the faces 112 has the same face circumferential width 132 and each of the ribs 130 has the same rib circumferential width 134. The rib circumferential width 134 is measured at a rib base region 130a of the rib 130 (e.g., where the rib 130 intersects the face 112). The face circumferential width 132 is measured adjacent the face 112 surface. In some embodiments, the face circumferential width 132 is about 2 mm or in a range from about 0.03 mm to about 4.1 mm and the rib circumferential width 134 is about 0.6 mm or in a range from about 0.4 mm to about 0.8 mm Thus, in these embodiments, a ratio of the face circumferential width 132 to the rib circumferential width 134 is about 3.0 or about 4.0 or in a range from about 0.1 to about 10.0 or from about 2.0 to about 10.0 or from about 3.0 to about 10.0. Thus, this design of the grip region 110 is distinct from the conventional cardboard applicator 10′ (FIG. 1C) with about a 1:1 ratio between the impression 40′ circumferential width and the outer surface 42′ circumferential width. The inventors recognized that this alternate ratio in the disclosed cardboard applicator 100 enhances the grip of the user on the grip region and thus advantageously enhances the user control of the applicator during use. The rib circumferential width 134 may be variable from the rib base region 130a to the rib peak region 130b. Stated another way, the rib 130 may be tapered from the rib base region 130a to the rib peak region 130b such that the rib base region 130a has a greater width than the rib peak region 130b. In some embodiments, the rib circumferential width 134 may be substantially the same in the rib peak region 130b and the rib base region 130a.

In some embodiments, the forming members 192 of the machine 190 are selected so to achieve one or more of the desired values and/or ratios of the face circumferential width 132 and the rib circumferential width 134. In some embodiments, the circumferential width of the face impression region 198 (FIG. 3E) of the forming member 192 is selected to be approximately equal to or within a certain range (e.g., ±20%) of a desired value of the face circumferential width 132. In other embodiments, the reduced circumferential spacing 214′ (FIG. 3E) of the forming members 192 in the fully compressed position is selected to be approximately equal to or within a certain range (e.g., ±20%) of a desired value of the rib circumferential width 134. In these example embodiments, a ratio of the circumferential width of the face impression region 198 to the reduced circumferential spacing 214′ is about equal to the desired ratio of the face circumferential width 132 to the rib circumferential width 134.

In other embodiments, as shown in FIG. 4D each of the circumferential faces 112 has an external face radius 131 and each of the ribs 130 has external rib radius 133. Along at least a portion of the face, the external rib radius 133 is greater than the external face radius 131. Each of the faces 112 may have the same external face radius 131 and each of the ribs 130 may have the same external rib radius 133. In some embodiments, the external rib radius 133 is about 7.0 mm or in a range from about 6.0 mm to about 10.0 mm and the external face radius 131 is about 3.5 mm or in a range from about 3.0 mm to about 7.0 mm. Thus, in these embodiments, a ratio of the external rib radius 133 to the external face radius 131 is about 2.0 or in a range from about 0.8 to about 3.0. Thus, this is distinct from the conventional cardboard applicator 10′ (FIG. 1C) where the ratio is much smaller which reduces the ability of the user to engage and grip the grip region of the applicator. The inventors recognized that this larger ratio in the disclosed cardboard applicator 100 enhances the grip of the user on the grip region (e.g., by providing sufficient radial space to position a user's finger so to grip one or more ribs 130) and thus advantageously enhances the user control of the applicator during use. In some embodiments, a distance between the peak region 130b of a first rib 130 and the peak region 130b of a second rib 130 that is radially opposite of the first rib 130 is about 17 mm or in a range from about 15 mm to about 20 mm.

In some embodiments, the machine 190 is configured so to achieve one or more of the desired values and/or ratios of the external rib radius 133 and the external face radius 131. As previously discussed, when operating the machine 190 the forming members 192 are moved from an initial radial position (FIG. 3C) in an inner radial direction 196 to a final radial position (FIG. 3E). The radial extent of movement of the forming members 192 from the initial radial position to the final radial position is selected so to achieve a desired value of the external face radius 131. Thus, in one example embodiment, to achieve a reduced value of the external face radius 131 the forming members 192 would be moved in the inner radial direction 196 to a final radial position that is more radially inward. One of ordinary skill in the art would then appropriately configure the machine 190 so that the final radial position is properly located so to achieve the desired value of the external face radius 131. In another embodiment, to achieve a desired value of the external rib radius 133, one or more of the magnitude of the inward radial pressure 202 applied by the forming members 192; the initial circumferential spacing 214 during the initial compression; the final circumferential spacing 214′ during the final compression and a shape or contour of the rib compression region 199 (FIG. 3E) are adjusted.

FIG. 4E is a cross-sectional view of a rib of the cross-sectional view of the grip region 110 of FIG. 4A. As shown in FIG. 4E, in some embodiments, the rib 130 includes a first rib portion 137 and a second rib portion 139 that are compressed or squeezed together (e.g., by the forming members 192 of the compression device 190) to form the rib 130. In some embodiments, a first internal rib surface 136 of the first rib portion 137 is in contact with a second internal rib surface 138 of the second rib portion 139. In some embodiments, the first internal rib surface 136 contacts the second internal rib surface 138 over at least a portion (e.g., greater than about 5% or greater than about 10% or greater than about 15%) of a rib height RH of the rib 130. In some embodiments, the first internal rib surface 136 contacts the second internal rib surface 138 over at least a majority (e.g., greater than 50%) of a height RH of the rib 130. In other embodiments, the first internal rib surface 136 contacts the second internal rib surface 138 over the entire height RH of the rib 130. The inventors of the present disclosure recognized that having these internal rib surfaces 136, 138 contact each other over at least a portion of the rib height RH maximizes the face circumferential width 132 and/or minimizes the rib circumferential width 134. Consequently, as previously discussed, this advantageously enhances the user control over the grip region 110 by enhancing the ability of the user to grip the barrel region 102.

FIG. 4F is a cross-sectional view of a rib of the cross-sectional view of the grip region 110. As shown in FIG. 4F, in some embodiments, the rib 130 includes a first rib portion 137 and a second rib portion 139 that are compressed or squeezed together (e.g., by the forming members 192 of the compression device 190) to form the rib 130. In some embodiments, a first internal rib surface 136 of the first rib portion 137 is in separated from a second internal rib surface 138 of the second rib portion 139 by a rib angle Θ. The rib angle Θ may be from about 0 degrees to about 15 degrees or from about 0 degrees to about 10 degrees, including all 0.1 degree increments form therein and thereby the recited range. The inventors of the present disclosure recognized that minimizing the distance between the internal rib surfaces 136, 138 maximizes the face circumferential width 132 and/or minimizes the rib circumferential width 134. Stated another way, the inventors of the present disclosure recognized that angling the internal rib surfaces 136, 138 with respect to one another maximizes the face circumferential width 132 and/or minimizes the rib circumferential width 134. Consequently, as previously discussed, this advantageously enhances the user control over the grip region 110 by enhancing the ability of the user to grip the barrel region 102. The amount of rebound or separation between the first internal rib surface 136 and the second internal rib surface 138 will be dependent on the aggressiveness (ratio of external rib radius 133 to external face radius 131). The larger the difference between these radii, the more likely there is to be a gap since the stress is concentrated on the tips of the rib and the fold at the base of the rib, causing the rib base to separate. As previously discussed, in some embodiments the external rib radius 133 and external face radius 131 are based on one or more parameters of the machine 190 (e.g., radial extent of movement of the forming members 192, applied pressure 202 of each forming member 192 in the inner radial direction) and/or of the forming members 192 (e.g., initial circumferential spacing 214, final circumferential spacing 214′, shape or contour of the rib compression region 199). Thus, in these embodiments, the amount of rebound is also based on one or more of these parameters. Furthermore, as previously discussed, the extent of rebound can also be based on parameters of the layers of the tube 194 (e.g., whether the adhesive has or has not cured when the tube 194 is radially compressed, which may reduce or increase the extent of the rebound).

FIG. 4G is a cross-sectional view of the grip region 110 of FIG. 4C taken along the line 4G-4G. As shown in FIG. 4G, in some embodiments each of the circumferential faces 112 of the grip region 110 project inwardly toward the longitudinal applicator axis 108.

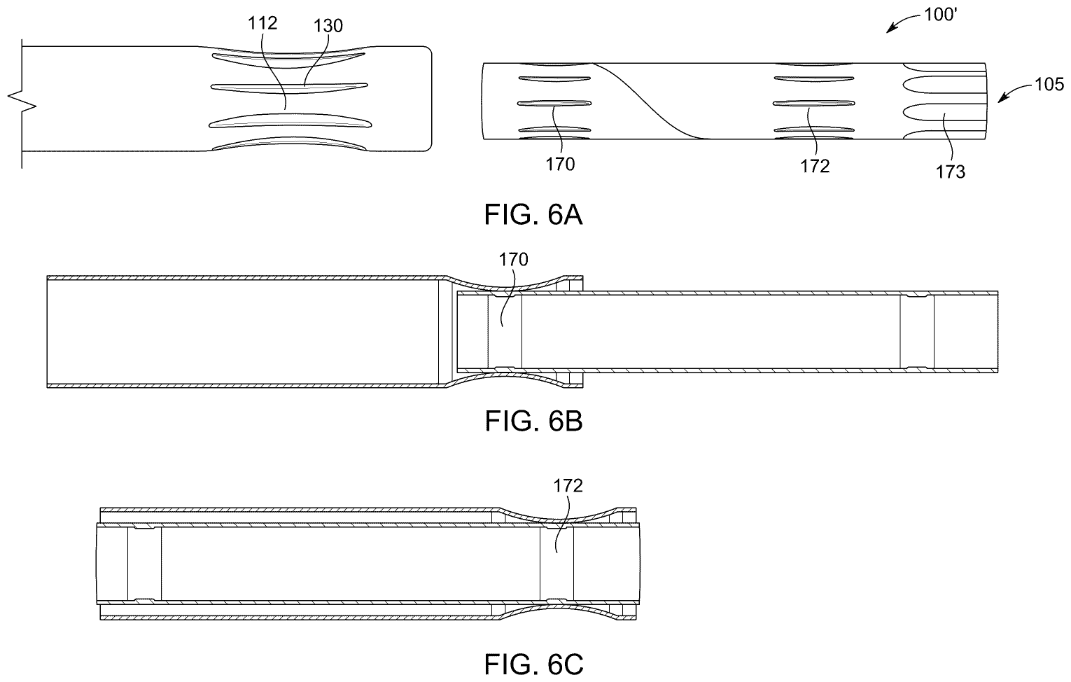

As further shown in FIG. 4G, the grip region 110 has a first internal grip diameter 124 and a second internal grip diameter 126, where the first internal grip diameter 124 is greater than the second internal grip diameter 126. Thus, in these embodiments, the grip region 110 features different diameters at different portions along the longitudinal applicator axis 108. In some embodiments, the first internal grip diameter 124 is about 13.3 mm or in a range from about 9.0 mm to about 20.0 mm and the second internal grip diameter 126 is about 6.0 mm or in a range from about 5.0 mm to about 14.0 mm. In some embodiments, a ratio of the second internal grip diameter 126 to the first internal grip diameter 124 is less than about 90%. The difference between the first internal grip diameter and the second internal grip diameter may be at least about 1 mm or at least about 1.5 mm or at least about 2 mm or at least about 2.5 mm or at least about 3 mm or at least about 3.5 mm or at least about 4 mm or at least about 5 mm or from about 1.5 mm to about 13 mm or from about 2.5 mm to about 10 mm. Thus, the circumferential faces 112 extend within the interior of the barrel region 102 towards the longitudinal applicator axis 108 by a greater extent than the impressions 40′ in the conventional cardboard applicator 10′ (FIG. 1C). The inventors of the present disclosure recognized that this advantageously ensures tight engagement between the plunger 104 and the grip region 110. The disclosed applicator 100 herein features the external plunger diameter 122 being noticeably less than the external barrel diameter 120 (FIG. 2A). The external plunger diameter 122 is measured (or defined) where the flange 161 (FIGS. 2C and 2D) or marking in regions 170, 172, 173 (FIG. 6A) are not present. The inventors of the present disclosure recognized that this ratio between the second internal grip diameter 126 and first internal grip diameter 124 facilitates tight engagement between the plunger 104 and the barrel region 102. In some embodiments, the second internal grip diameter 126 is at least 30% of the first internal grip diameter 124. In some embodiments, a difference between the first internal grip diameter 124 and the second internal grip diameter 126 is at least about 1 mm and/or about 2.5 mm and/or about 3 mm and/or about 3.5 mm and/or about 4 mm or from about 1 mm to about 15 mm.

In some embodiments, one or more parameters of the machine 190 and forming members 192 can be adjusted, to achieve a desired value of the difference between the first internal grip diameter 124 and the second internal grip diameter 126. In one example embodiment, a difference between the initial radial position (FIG. 3C) when initially compressing the tube 194 and a final radial position (FIG. 3E) when finally compressing the tube 194 can be adjusted so to achieve the desired value of the difference between the first and second internal grip diameters 124, 126.

The inventors of the present disclosure designed the grip region 110 so that the barrel region 102 may support the plunger 104 at multiple regions along the longitudinal applicator axis 108. As shown in FIG. 4G, in some embodiments the plunger 104 is supported by the grip region 110 (at the second internal grip diameter 126) and is also supported at the rearward end region 129 of the barrel region 102. As illustrated in FIGS. 4C and 4G, the rearward end region 129 of barrel region 102 may be inverted or rolled such that an external surface of the rearward end is configured to become an internal surface and engage a portion of the plunger 104. The external plunger diameter 122 of the plunger 104 (FIG. 2A) is greater than 90% of the second internal grip diameter 126 of the grip region 110. The inventors of the present disclosure recognized that this facilitates tight engagement between the plunger 104 and the grip region 110 while still permitting the plunger 104 to slide within the barrel region 102 relative to the grip region 110. For purposes of this disclosure, “tight engagement” corresponds to a tolerance (e.g., difference between the external plunger diameter 122 and the second internal grip diameter 126) that is about 0.3 mm to about 0.5 mm and/or about 0.2 mm to about 0.6 mm and/or about 0.1 mm to about 0.8 mm.

As shown in FIG. 4G, in some embodiments the second internal grip diameter 126 of the grip region 110 contacts the plunger 104 over a minimum longitudinal length 162 along the longitudinal applicator axis 108. The minimum longitudinal length 162 may be at least about 12 mm or at least about 15 mm or in a range from about 12 mm to about 20 mm along the longitudinal applicator axis 108. As shown in FIG. 4G in some embodiments the circumferential faces 112 of the grip region 110 are substantially flat over the minimum longitudinal length 162. In some embodiments, one or more parameters of the forming members 192 are selected so to achieve a desired value of the minimum longitudinal length 162. In one example embodiment, the face impression region 198 (FIG. 3E) of the forming members 192 is selected to have a profile in the longitudinal direction (e.g., parallel to axis 108 and perpendicular to the plane of FIG. 3E) which resembles the desired longitudinal profile of the circumferential face 112 along the longitudinal applicator axis 108. Thus, in one example embodiment, the face impression region 198 includes a flat surface along the longitudinal direction that is about equal to or within a certain range (±20%) of the minimum longitudinal length 162.

As further shown in FIG. 4G, in some embodiments the grip region 110 of the barrel region 102 contacts the plunger 104 at a first contact point 165 and the rearward end region 129 of the barrel region 102 contacts the plunger 104 at a second contact point 167. The contact points 165, 167 are spaced apart along the longitudinal applicator axis 108 by a distance 164. The distance 164 is about 8 mm or in a range from about 6 mm to about 10 mm. The inventors of the present disclosure recognized that providing multiple contact points 165, 167 between the plunger 104 and the barrel region 102 advantageously helps to stabilize the plunger 104 as it is inserted into and retracted from the barrel region 102. The inventors of the present disclosure also recognized that spacing the contact points 165, 167 by a distance disclosed herein further facilitates this stabilization.

As further shown in FIG. 4G, in some embodiments each of the circumferential faces 112 extends from an upper grip region 152 to a lower grip region 154. In some embodiments, the faces 112 have a radius of curvature 156 in one or both of the regions 152, 154 that is about 8.3 mm or in a range from about 0.1 mm to about 425 mm. In some embodiments, the forming members 192 are shaped so to achieve a desired value of this radius of curvature 156 in the upper and lower grip region 152, 154. In one example embodiment, the radius of curvature of the face impression region 198 (FIG. 3E) along the longitudinal axis 108 is shaped so to have a radius of curvature that is about equal to or within a certain range (±20%) of the radius of curvature 156 of the regions 152, 154 and a flat region therebetween having a longitudinal length that is about equal to or within a certain range (±20%) of the minimum longitudinal length 162. The radius of curvature may be determined by taking the (face length)²/(face depth), where face length is the length of the face 112 along the longitudinal applicator axis 108 and the face depth is a depth of the face 112 measured perpendicular to the longitudinal applicator axis 108 (e.g., the difference between the external rib radius 133 and the external face radius 131). The face length may be from about 3 mm to about 30 mm and the face depth may be from about 0.1 mm to about 3 mm. For example, the face length may be about 25 mm and the face depth may be about 2 mm. In some embodiments, the radii of curvature is the same for each of the circumferential faces 112. In this example embodiment, the radius of curvature 156 is measured in a plane perpendicular to the longitudinal applicator axis 108. The inventors of the present disclosure recognized that having the radius of curvature 156 in this range advantageously maximizes the length of the circumferential faces 112 (e.g., along the longitudinal applicator axis 108) which further enhances the user ability to grip the grip region 110 and thus maximizes user control of the barrel region 102. Although FIG. 4G depicts that the grip region 110 is substantially cylindrical between the regions 152, 154 in other embodiments the grip region 110 is concave or convex between the regions 152, 154.

It is also to be appreciated that the rearward end region 129 may not contact the plunger 104. The rearward end region 129 may be shaped such that it extends outward away from the plunger 104, or stated another way, there is a gap between the plunger 104 and the rearward end region 129. In those embodiments where the reward end region 129 does not contact the plunger 104, the face 112 may engage or contact the plunger 104 along the minimum longitudinal length 162, as previously discussed.

As illustrated in FIG. 4H, in some embodiments the plunger 104 is supported by the grip region 110 (at the second internal grip diameter 126) and is also supported at the rearward end 129′ of the outer member. As illustrated in FIGS. 4A and 4H, the rearward end region 129′ of may be configured to engage a portion of the plunger 104. The external plunger diameter 122 of the plunger 104 (FIG. 2A) is greater than 90% of the second internal grip diameter 126′ of the grip region 110. The inventors of the present disclosure recognized that this facilitates tight engagement between the plunger 104 and the grip region 110 while still permitting the plunger 104 to slide within the barrel region 102 relative to the grip region 110.

As shown in FIG. 4H, the second internal grip diameter 126′ of the grip region 110 contacts the plunger 104 over a minimum longitudinal length 162′ along the longitudinal applicator axis 108. The minimum longitudinal length 162′ may be at least about 2 mm or at least about 5 mm or at least about 7 mm or in a range from about 2 mm to about 10 mm along the longitudinal applicator axis 108. As shown in FIG. 4H, in some embodiments, the circumferential faces 112 of the grip region 110 are substantially curved over the minimum longitudinal length 162′. In some embodiments, the face impression region 198 of the forming members 192 is shaped in the longitudinal direction along the axis 108 so to achieve a desired value of the minimum longitudinal length 162′. In this example embodiment, since the minimum longitudinal length 162′ is less than the minimum longitudinal length 162 of FIG. 4G, the flat region of the face impression region 198 in the longitudinal direction has a reduced value as compared to the flat region of the face impression region 198 used to form the minimum longitudinal length 162 of the circumferential face 112 of FIG. 4G.

As further shown in FIG. 4H, in some embodiments the grip region 110 of the barrel region 102 contacts the plunger 104 at a first contact point 165′ and the rearward end 129′ of the barrel region 102 contacts the plunger 104 at a second contact point 167′. The contact points 165′, 167′ are spaced apart along the longitudinal applicator axis 108 by a distance 164′. The distance 164′ is about 8 mm or in a range from about 6 mm to about 10 mm. The inventors of the present disclosure recognized that providing multiple contact points 165′, 167′ between the plunger 104 and the barrel region 102 advantageously helps to stabilize the plunger 104 as it is inserted into and retracted from the barrel region 102. The inventors of the present disclosure also recognized that spacing the contact points 165′, 167′ by a distance disclosed herein further facilitates this stabilization.

As further shown in FIG. 4H, each of the circumferential faces 112 extends from an upper grip region 152 to a lower grip region 154. In some embodiments, the faces 112 have a radius of curvature 156′ in one or both of the regions 152, 154 that is about 300 mm or in a range from about 4.3 mm to about 900 mm. As with the embodiment of FIG. 4G, one or more parameters of the face impression region 198 of the forming members 192 are adjusted so to achieve a desired value of the radius of curvature 156′ in the regions 152, 154. In some embodiments, the radius of curvature of the face impression region 198 along the longitudinal direction would be selected to be about equal to or within a certain range (±20%) of the desired value of the radius of curvature 156′. The radius of curvature 156′ is determined by taking the (face length)²/(face depth). The face length may be from about 3 mm to about 30 mm and the face depth may be from about 0.1 mm to about 3 mm. For example, the face length may be about 25 mm and the face depth may be about 2 mm. In some embodiments, the radii of curvature 156′ is the same for each of the circumferential faces 112. In this example embodiment, the radius of curvature 156′ is measured in a plane perpendicular to the longitudinal applicator axis 108. The inventors of the present disclosure recognized that having the radius of curvature 156′ in this range advantageously maximizes the length of the circumferential faces 112 (e.g., along the longitudinal applicator axis 108) which further enhances the user ability to grip the grip region 110 and thus maximizes user control of the barrel region 102. Although FIG. 4H depicts that the grip region 110 is substantially concave between the regions 152, 154, the grip region 110 may be substantially flat, concave, and/or convex between the regions 152, 154.

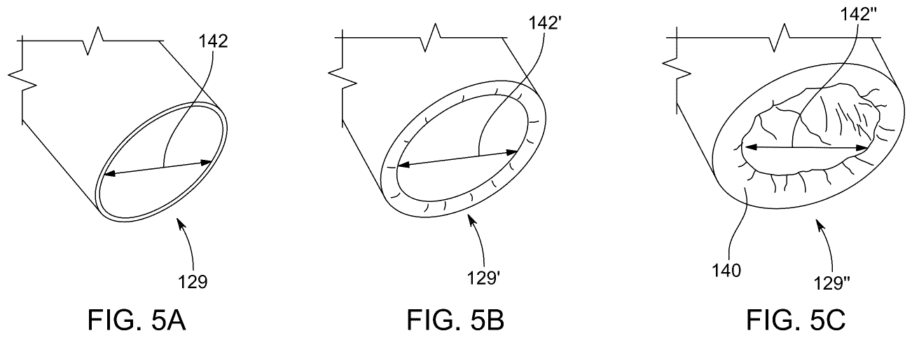

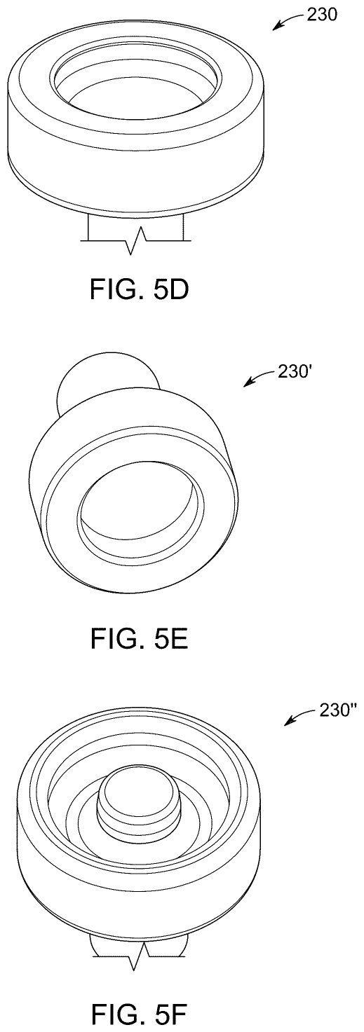

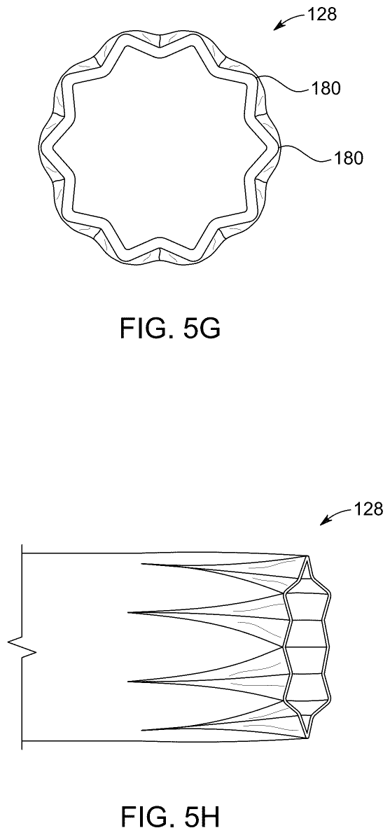

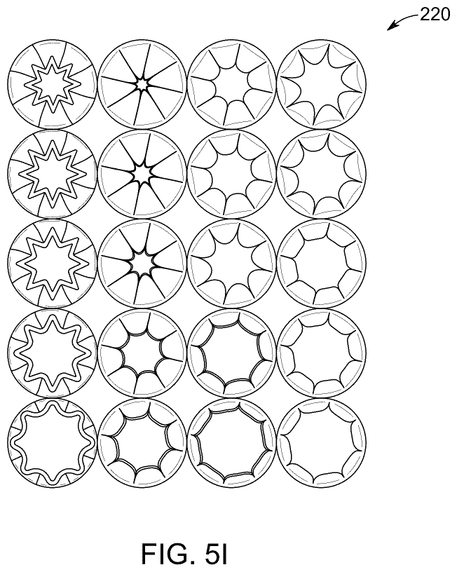

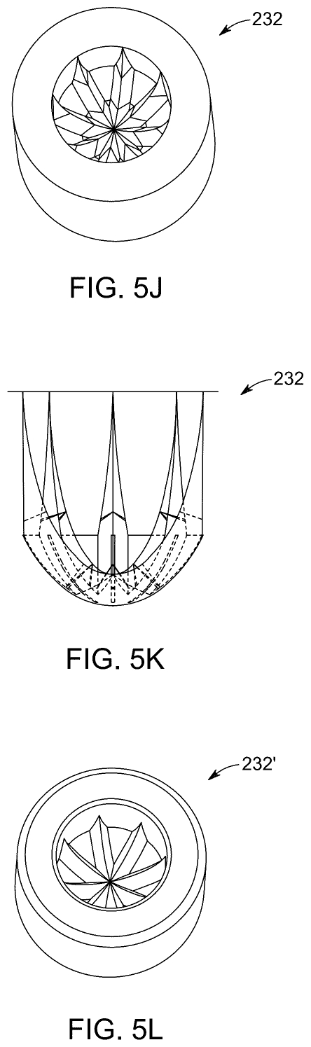

FIGS. 5A through 5C are perspective views of the rearward end region 129 of the applicator 100 of FIG. 2A. As shown in FIGS. 5A through 5C, the rearward end region 129 is disposed adjacent to the grip region 110. The rearward end region 129 comprises an internal end region diameter 142. FIG. 5A shows the rearward end 129 with the internal end region diameter 142 without any modification to the rearward end 129. FIGS. 5B and 5C depict respective rearward end regions 129′, 129″ where the rearward end region has been modified (e.g., by rolling) so to reduce the internal end region diameter 142′, 142″ relative to the internal end region diameter 142 in FIG. 5A. In these embodiments, the internal end region diameters 142′, 142″ are each less than the internal barrel diameter 144. In some embodiments, where the second contact points 167, 167′ of FIGS. 4G and 4H is between the plunger 104 and the rearward end 129″ of FIG. 5C with the internal end region diameter 142″, the external plunger diameter 122 is at least 90% of the internal end region diameter 142″. This facilitates the same tight engagement at the second contact point 167, 167′ as was discussed previously with respect to the first contact point 165. In some embodiments, the rearward end 129″ of FIG. 5C includes a rolled edge 140 that has the internal end region diameter 142″. The difference between the rearward end 129′ of FIG. 5B and rearward end 129″ of FIG. 5C is the degree to which the rearward end has been rolled. FIG. 5C is rolled to a greater extent in the rearward end 129″. The inventors of the present disclosure recognized that the rolled edge 140 advantageously results in a smaller internal end region diameter 142″ which can aid in stabilizing the plunger 104 particularly at the rearward end 129 of the barrel region 102. Without the rolled edge 140 of the rearward end 129″, the plunger 104 would only contact the interior surface of the barrel region 102 at the first contact point 165 rather than at both contact points 165, 167 and thus there would not be as much stability between the plunger 104 and barrel region 102 as the plunger 104 is moved into and out of the outer member 103. As shown in FIGS. 5A through 5C, each of the end regions 129′, 129″ are tapered inwardly in an inner radial direction.

FIGS. 5D through 5F are perspective views of various curling tools 230, 230′, 230″ that are used to compress the end regions 129, 129′, 129″ of FIGS. 5A through 5C. Unlike the forming members 192 of the machine 190 which compress the outer tube 194 in the inner radial direction 196, the curling tools 230, 230′, 230″ compress the rearward end region 129 of the tube 194 in the longitudinal direction. One or more parameters of the tool 230, 230′, 230″ are selected so to achieve desired values of the rolled end regions 129, 129′, 129″. In one example embodiment, the tool 230 of FIG. 5D is selected to roll the end region 129′ of FIG. 5B having the internal end region diameter 142′ whereas the tool 230″ of FIG. 5F is selected to roll the end region 129″ of FIG. 5C having the internal end region diameter 142″ that is smaller than the internal end region diameter 142′ of the end region 129′ of FIG. 5B. The tool 230″ of FIG. 5F features an interior region within the tool that has an increased radius of curvature as compared to the interior region of the tool 230 of FIG. 5D and thus consequently rolls the end region 129″ with a reduced internal end region diameter 142″.

In order to form the end region 129 having one or more desired characteristics, the appropriate tool 230, 230′, 230″ is selected to longitudinally compress the end region 129 so to have these desired characteristics. In one example embodiment, these desired characteristics include the rolled end region 129 providing the contact point 167 at the second internal grip diameter 126 (FIG. 4G). Thus, the appropriate tool 230, 230′, 230″ is selected to longitudinally compress the end region 129 resulting in the depicted longitudinal profile of the rolled end region in FIG. 4G where the end region 129 forms the second contact point 167 at the second internal grip diameter 126.

The forward end region 128 of the outer member 103 is now discussed. FIGS. 5G through 5I are perspective views of a forward end region of the applicator 100 of FIG. 2A. FIGS. 5G and 5H depict various views of the pleats 180 180 formed at the forward end region 128. FIG. 51 depicts various arrangements of pleats 180 formed in the forward end region 128.

As with the rearward end region 130, the forward end region 128 is compressed by a tool in a longitudinal direction (e.g., in an opposite direction as the direction of compression of the rearward end region 130). FIGS. 5J through 5L are perspective views of various pleating tools 232, 232′, 232″ that are used to compress the forward end regions 128 of FIGS. 5G through 5I. As shown in FIGS. 5J through 5L, the pleating tool 232, 232′, 232″ features an interior chamber with a number of wedge or triangular shaped members spaced apart in a circumferential direction. Upon compressing the forward end region 128 of the barrel region 102 with the pleating tool 232, 232′, 232″ the pleats 180 are formed. In some embodiments, the number of pleats 180 formed in the forward end region 128 is based on the number of wedge or triangular shaped spaced apart members of the pleating tool 232, 232′, 232″. Thus, the pleating tool 232, 232′, 232″ used to compress the forward end region 128 is selected based on the number of wedge shaped members. Additionally, in forming the pleats 180 at the forward end region 128, the extent to which the pleats 180 are curved inwardly at the forward end region 128 depends on various factors such as the longitudinal pressure exerted by the pleating tool 232, 232′, 232″ on the forward end region 128. Thus, in one example embodiment, FIG. 51 depicts that for each horizontal row of forward end regions 128, those forward end regions 128 on the right side of FIG. 5I are longitudinally compressed by a greater extent and thus the petals 180 are more inwardly directed towards the right side of each horizontal row of FIG. 51. The forward end region 128 may also be slit or cut to form two or more petals and longitudinally compressed to curve the petals inwardly. The forward end region 128 may be cut or slit prior to forming the grip region or subsequent to forming the grip region.

Although the previous embodiments discuss that the machine 190 can be used to radially compress the grip region 110 in the outer member 103 of the applicator 100, in other embodiments the machine 190 is used to radially compress the inner member 105 (e.g., plunger 104). FIGS. 6A through 6C are side views of an applicator 100′ including a barrel region and a plunger with circumferential faces formed in both the barrel region and the plunger. As illustrated in FIG. 6A, the plunger 104 features circumferential faces and/or other markings formed in multiple engagement regions 170, 172, 173. The circumferential faces formed in the multiple engagement regions 170, 172, 173 of the plunger 104 provide physical faces that may cause the plunger 104 to advance differently as the plunger is inserted into the barrel region 102. For example, these multiple engagement regions 170, 172, 173 may result in areas of increased or decreased friction as the plunger advances into the barrel. However, these regions of the plunger 104 are moderate and can be easily overcome by the user applying additional force or less force to the plunger 104. In some embodiments, such as in FIG. 6B, the plunger 104 inserted within the barrel region 102 until the circumferential faces in the first engagement region 170 engage the grip region 110 of the barrel region 102. FIG. 6C then depicts the plunger 104 after being inserted further within the barrel region 102 until the circumferential faces in the second engagement region 172 engage the grip region 110 and the circumferential faces in the third engagement region 173 are positioned at the end region 129 of the barrel region 102. The circumferential faces (or markings) formed in the multiple engagement regions 170, 172, 173 of the plunger 104 assist the user when inserting the plunger 104 into the barrel region 102 by providing either physical indicators (circumferential faces) or visual indicators (markings) to confirm when the plunger 104 has reached various phases of insertion within the barrel region 102. In the embodiments where circumferential faces are formed in the multiple engagement regions 170, 172, 173 of the plunger 104, these circumferential faces are formed in a similar manner (e.g., using the compression device 190 of FIGS. 3A and 3B) as the circumferential faces formed in the barrel region 102.

The multiple engagement regions 170, 172, 173 of the inner member 105 (e.g., plunger 104) are formed with the machine 190 using a similar process that was used to form the grip region 110 of the barrel region 102. In these embodiments, the outer tube 194 is selected based on various desired characteristics of the plunger 104 (e.g., outer diameter 122). The forming members 192 of the machine 190 are then selected so that the profiles of the forming members 192 form the desired profile of the engagement portions 170, 172, 173. In some embodiments, multiple machines 190 are used to simultaneously form the multiple engagement portions 170, 172, 173 in the plunger 104 whereas in other embodiments the same machine 190 compresses the plunger 104 at different times at different regions along the length of the plunger 104 to form the multiple engagement portions 170, 172, 173. In these embodiments, the forming members 192 of the machine 190 are selected such that upon radially compressing the plunger 104, the first engagement portion 170 is formed with a specific profile that is configured to operatively engage the grip region 110 when the plunger 104 is positioned in a pre-dispensing position (FIG. 6B). In other embodiments, the forming members 192 of the machine 190 are selected such that upon radially compressing the plunger 104, the second engagement portion 172 is formed with a specific profile that is configured to operatively engage the grip region 110 upon movement of the plunger 104 to a post-dispensing position (FIG. 6C).

The layer structure of the tube 194 and thus the layer structure of the outer member 103 (e.g., barrel region 102) formed using the tube 194 is now discussed. In other embodiments, the tube 194 is used to form the inner member 105 (e.g., plunger 104) and thus for those embodiments the layer structure of the inner member 105 is also now discussed. The outer member 103 may be formed from one or more layers of material, such as paper. FIG. 7A is a cross-sectional side view of a plurality of material layers 146a through 146d of the tube 194 that is used to form one or more components that define the applicator 100. Although four layers 146a through 146d are depicted in FIG. 7A, in other embodiments less or more than four layers may define the applicator 100. For example, FIG. 7D illustrates an embodiment with two layers 146a and 146b and one or more layers of a coating 147 and/or an adhesive 149. The one or more of the layers 146a through 146d may exclude non-naturally occurring fibers. The one or more layers 146a through 146d may include naturally-occurring fibers. Naturally occurring fibers may include cellulosic fibers, wool, silk and other naturally-occurring protein fibers and mixtures thereof, cotton, cotton linters, flax, sisal, abaca, hemp, hesperaloe, jute, bamboo, bagasse, kudzu, corn, sorghum, gourd, agave, loofah, trichomes, seed-hairs, wheat, and mixtures thereof. In other embodiments, each of the one or more layers 146a through 146d include cellulosic material including wood fibers. Wood fiber(s) means fibers derived from both deciduous trees (hereinafter, also referred to as “hardwood”) and coniferous trees (hereinafter, also referred to as “softwood”) may be utilized. Wood fibers may be short (typical of hardwood fibers) or long (typical of softwood fibers). Nonlimiting examples of short fibers include fibers derived from a fiber source selected from the group consisting of Acacia, Eucalyptus, Maple, Oak, Aspen, Birch, Cottonwood, Alder, Ash, Cherry, Elm, Hickory, Poplar, Gum, Walnut, Locust, Sycamore, Beech, Catalpa, Sassafras, Gmelina, Albizia, Anthocephalus, and Magnolia. Nonlimiting examples of long fibers include fibers derived from Pine, Spruce, Fir, Tamarack, Hemlock, Cypress, and Cedar. The one or more of the layers 146a through 146d may be free of synthetic fibers and films.

FIG. 7B is a side view of an applicator including the material layers 146a through 146d of FIG. 7A spirally wound to form the barrel region 102. In some embodiments, the one or more layers 146a through 146d are spirally wound to form at least one of the barrel region 102 and/or plunger 104. FIG. 7B depicts the spiral or non-linear seam 150 formed when the layers 146a through 146d are spirally wound to form the barrel region 102. In some embodiments, one or more of the layers 146a through 146d can include other materials, such as plastic. However, in other embodiments, each of the layers 146a through 146d are 100% cellulosic. In some embodiments, the cellulosic material of the one or more layers 146a through 146d is paper. In other embodiments, the cellulosic material of the one or more layers 146a through 146d is pulp.

FIG. 7C is a side view of an applicator including the material layers 146a through 146d of FIG. 7A convolutely wound to form the barrel region 102. In some embodiments, the one or more layers 146a through 146d are convolutely wound to form at least one of the barrel region 102 and/or plunger 104. FIG. 7C depicts the linear seam 151 formed when the layers 146a through 146d are convolutely wound to form the barrel region 102. In some embodiments, one or more of the layers 146a through 146d can include other materials, such as plastic. However, in other embodiments, each of the layers 146a through 146d are 100% cellulosic. In some embodiments, the cellulosic material of the one or more layers 146a through 146d is paper. In other embodiments, the cellulosic material of the one or more layers 146a through 146d is pulp.

The outer member 103 may be provided in the form of a spirally wound, convolutely wound or other seamed hollow tube which may be formed from paper, paperboard, cardboard or a combination thereof. The paper, paperboard, or cardboard may be manufactured predominantly of wood and/or cellulose pulp fiber. The barrel region 102 may be manufactured to be relatively rigid and have an external barrel diameter 120 of about 10 millimeters to about 20 millimeters. The barrel region 102 has a wall which may be manufactured to have a predetermined thickness of about 0.1 mm to about 0.7 mm. The wall may be constructed from a single layer of material or be formed from two or more plies or layers that are bonded together to form a laminate.

The use of two or more plies or layers enables the manufacturer to use particularly selected materials in the various layers that may enhance the performance of the tampon applicator 100. When two or more plies are included, all the plies may be spirally wound, convolutely wound, and/or longitudinally seamed to form an elongated cylinder. The wall may be constructed using a relatively smooth thin layer of material on the outside or exterior surface that surrounds a relatively coarser and possibly thicker layer. When the wall includes at least three layers, the middle layer may be the thicker layer and the interior and exterior layers may be imparted with a relatively smooth and/or slippery surface finish (e.g., via application of a coating) to facilitate expulsion of the tampon and to facilitate comfortable insertion of the barrel region 102 into the vaginal cavity, respectively. By sandwiching a thick, coarser layer of material between two thin, smooth layers, a barrel region 102 may be provided which is economical and functional. The wall may include one to three, four or five layers, although more layers may be utilized if desired.

The plies, or layers, forming the wall may be held together by a coating, such as an adhesive, or by a coating activated by heat and/or pressure, or by mechanical interaction such as by ultrasonic bonding or pressure bonding, or a combination thereof. The coating may be a petroleum, a petroleum derivative, and/or a plastic with petroleum or petroleum derived components or precursor materials. Thus, an applicator may comprise less than 10 percent by weight or less than 5 percent by weight petroleum, petroleum derivatives and/or plastics with petroleum or petroleum-derived components or precursor materials. For example, each of the layers of the applicator may comprise a cellulosic material, such as paper, and the coating between the layers or plies may be a petroleum, a petroleum derivative, and/or a plastic with petroleum or petroleum derived components or precursor materials. Alternatively, the applicator may comprise a coating, such as an adhesive that is not a petroleum, a petroleum derivative, and/or a plastic with petroleum or petroleum derived components or precursor materials.

The coating may be either water-soluble or water-insoluble. For example, a water-soluble coating, such as a water-soluble adhesive, may be preferred, for reasons of environment-related concerns, so that the layers of the wall will readily separate when wetted by water. Such wetting may occur, for example, upon immersion by flushing in a toilet, or upon exposure to environmental moisture (for example, in an outside landfill in moist or wet conditions). When a water-soluble coating is used, exposure of the barrel region 102 to processes of a municipal waste treatment plant, wherein soaking in water, interaction with chemicals and agitation all occur, will cause the layers of the wall to separate and disperse in a relatively short period of time.

In addition to the objective of providing for dissolution and dispersibility, as noted above, it may be desirable to reduce or eliminate components derived from petroleum according to currently recognized objectives relating to use of sustainably-sourced and environmentally-friendly materials. Many (if not all) adhesives currently used to adhere layers of paper together to form tube structures for applicator components are aqueous formulations including polyvinyl acetate (PVAc). PVAc-based adhesive (exemplified by “white glue” or ELMER'S brand glue) is desirable because it is water soluble, readily penetrates pores of fibrous cellulose/wood pulp paper and adheres to cellulose fibers, is tacky when applied (promoting rapid setting and bonding), and can be formulated with relatively high water content and low viscosity for ease of application during manufacturing. However, PVAc is derived from hydrocarbons (acetylene or ethylene), most economically obtained from petroleum. Other non-petroleum-derived adhesive components might be considered, including hide glue and sodium silicate (water glass), used elsewhere in paper-related applications.

When tube stock used to form an applicator or ejection plunger is formed of paper (in turn, formed of cellulose pulp such as wood pulp), the paper may be relatively hydrophilic, porous and absorbent, and also, potentially abrasive to sensitive tissue prior to a coating being applied. The hydrophilic, porous and absorbent qualities may cause the paper material to draw moisture from moist tissues, and thereby increase the potential for irritation and abrasion from sliding contact therebetween. Additionally, these qualities may increase the potential for staining of the paper material with menstrual fluid, which, following withdrawal of an applicator from the vaginal cavity, some users may find undesirable. Accordingly, it may be desirable to apply a coating to the material that, when in melt and/or liquid form readily penetrates the fibrous structure of the paper and seals it to prevent it from absorbing fluid. The coating may be applied to one or more layers or plies, such as the outer layer of the barrel and/or the inner layer of the barrel. The coating may be applied to the layer or ply prior to forming the applicator. More specifically, the coating may be applied prior to the one or more layers being convolutely wound and/or spirally wound.

### Method to Form the Applicator

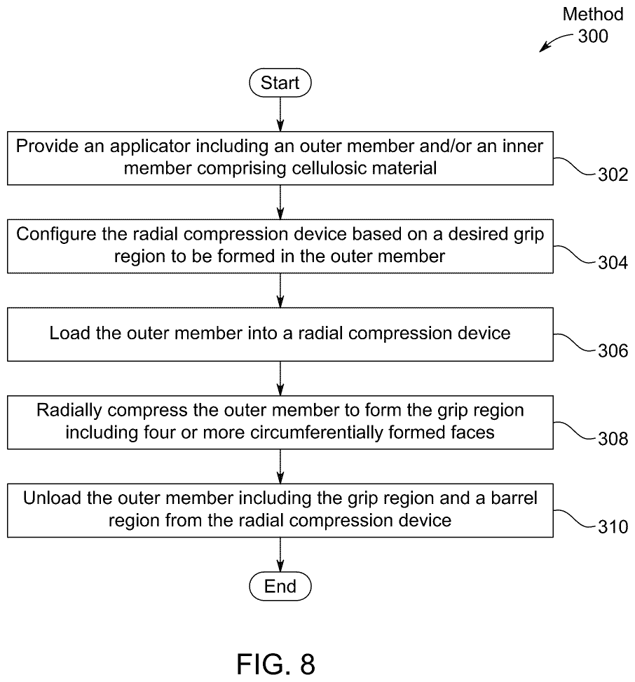

A flowchart depicting one or more steps of a method for forming the applicator 100 is now discussed. FIG. 8 is a flowchart depicting one or more steps of a method 300 for forming a tampon applicator 100. Although steps are depicted in FIG. 8 as integral steps in a particular order for purposes of illustration, in other embodiments, one or more steps, or portions thereof, are performed in a different order, or overlapping in time, in series or in parallel, or are omitted, or one or more additional steps are added, or the method is changed in some combination of ways.

In step 302, the applicator is provided including the outer member and the inner member. At least one of the outer member or inner member may comprise cellulosic material (e.g., external diameter 120). In another embodiment, in step 302 the tube 194 is provided that has one or more desired characteristics of the inner member 105 (e.g., external diameter 122).

In step 304 the compression device is configured based on a desired profile to be formed in the applicator. In some embodiments, in step 304 the compression device is the radial compression device 190 and the desired profile is the grip region 110 to be formed in the tube 194 to form the outer member 103 (e.g., barrel region 102). In other embodiments, in step 304 the desired profile is the one or more engagement regions 170, 172, 173 to be formed in the tube 194 to form the inner member 105 (e.g., plunger 104).

In step 304, one or more parameters of the machine 190 and/or the forming members 192 are selected in order to achieve a desired profile in the applicator by radially compressing the tube 194 with the forming members 192. In some embodiments, in step 304 the forming members 192 are selected based on their profile so that the forming members 192 compress the grip region 110 with a desired profile in the tube 194. Thus, in some example embodiments, in step 304 eight forming members 192 are selected with a profile similar to shown in FIG. 3E in order to compress a grip region 110 comprising eight circumferential faces 112 each spaced apart by a respective one of eight ribs 130. In some embodiments, as shown in FIGS. 3J and 3N the forming member 192 has multiple profiles on different ends thereof and thus in step 304 the forming member 192 is oriented such that the desired profile is aligned to compress the tube 194 and is then secured to the machine 190 prior to the compressing step.

In step 306, after configuring the machine 190 and/or forming members 192 to achieve the desired profile in the applicator, the tube 194 is loaded into the machine 190. As shown in FIG. 3B, the tube 194 is loaded into the machine 190 such that the forming members 192 are circumferentially arranged around the tube 194. In some embodiments, in step 306 the stabilizing rod or mandrel 118 is positioned within the interior portion 119 of the tube 194. However, in other embodiments the stabilizing rod or mandrel 118 is not utilized.

In step 308, after loading the tube 194 into the machine 190 in step 306, the tube 194 is radially compressed by the forming members 192 being moved in the inward radial direction 196. In some embodiments, in step 308 the forming members 192 are moved in the inner radial direction 196, initially compress the tube 194 at an initial compressed position (FIG. 3C) and continue to compress the tube 194 until reaching a final compressed position (FIG. 3E). In some embodiments, in step 308 four or more circumferential faces 112 and four or more ribs 130 are formed in the tube 194. In still other embodiments, in step 308 the tube 194 is radially compressed by the forming members 192 to form the one or more engagement regions 170, 172, 173 in the tube 194 in order to form the inner member 105 (e.g., plunger 104).

In other embodiments, in step 308 the tube 194 is compressed in a direction other than the inner radial direction. In one example embodiment, in step 308 one of the rearward end region 129 or forward end region 108 of the tube 194 are compressed in a longitudinal direction (e.g., parallel to the axis 108). In some embodiments, in step 308 one of the tools 230, 230′, 230″ is used to longitudinally compress the rearward end region 129 to form one or more of the rolled end regions 129, 129′, 129″. In other embodiments, in step 308 one of the pleated tools 232, 232′, 232″ is used to longitudinally compress the forward end region 128 to form one or more petals 180 in the forward end region 128 having desired characteristics (e.g., number of petals, shape of the petals, etc.).

In step 310, after radially compressing the tube 194 to form the desired profile in the outer member 103 (e.g., barrel region 102) or inner member 105 (e.g., plunger 104), the outer member 103 or inner member 105 are unloaded from the machine 190. In some embodiments, in step 310 after forming the grip region 110 in the outer member 103, the outer member 103 is unloaded form the machine 190. In still other embodiments, in step 310 after forming the engagement regions 170, 172, 173 in the inner member 105 (e.g., plunger 104), the inner member 105 is removed from the machine 190. In still other embodiments, where the parent applicator roll 206 is compressed in step 308, in step 310 the parent applicator roll 206 is unloaded from the machine 190 and subsequent steps (e.g., cutting the applicator parent roll 206 along the cut lines 107 into multiple outer members 103 each having the grip region 110) are performed.

Heat and/or steam and/or moisture may be applied to the one or more layers and/or the cylindrical segment to aid in forming the grip region. However, the increased moisture content during manufacture of the outer member and/or the inner member does not remain after formation of the outer member/inner member. The tampon applicator, including at least one of the outer member and the inner member, of the present invention may have a water (moisture) content (% water in sample) from 0% to about 20%; in certain embodiments, tampon applicator of the present invention may have a water (moisture) content from about 1% to about 15%; in certain embodiments, tampon applicator of the present invention may have a water (moisture) content from about 2% to about 10%; and in certain embodiments, tampon applicator of the present invention may have a water (moisture) content from about 4% to about 10%, wherein the water content is measured by the Water Content Test Method described herein.

### Test Method

### Water Content Test Method

The water (moisture) content present in a tampon applicator of the present invention is measured using the following Water Content Test Method. A tampon applicator or portion thereof (“sample”) is placed in a conditioned room at a temperature of 23° C.±1.0° C. and a relative humidity of 50%±2% for at least 24 hours prior to testing. Under the temperature and humidity conditions mentioned above, using a balance with at least four decimal places, the weight of the sample is recorded every five minutes until a change of less than 0.5% of previous weight is detected during a 10 minute period. The final weight is recorded as the “equilibrium weight”. Within 10 minutes, the samples are placed into the forced air oven on top of foil for 24 hours at 70° C.±2° C. at a relative humidity of 4%±2% for drying. After the 24 hours of drying, the sample is removed and weighed within 15 seconds. This weight is designated as the “dry weight” of the sample.

The water (moisture) content of the sample is calculated as follows:

$$\% Water in sample = 100 \% \times \frac{\begin{matrix}( Equilibrium weight of sample - \\ Dry weight of sample )\end{matrix}}{Dry weight of sample}$$

The % Water (moisture) in sample for 3 replicates is averaged to give the reported % Water (moisture) in sample. Report results to the nearest 0.1%.

### Combinations

The following combinations are contemplated herein:
A1. A method of forming a tampon applicator, the method comprising: providing an applicator comprising an outer member and an inner member, wherein each of the outer member and the inner member comprise cellulosic material; loading the outer member into a compression device; compressing the outer member to form a grip region, wherein the grip region comprises greater than four faces that are circumferentially formed about the outer member; and unloading the outer member comprising the grip region and a barrel region from the compression device.
A2. The method according to paragraph A1, wherein the compressing the outer member comprises radially compressing the outer member to form the grip region.
A3. The method according to paragraph A2, comprising inserting a stabilizing rod within an interior portion of the outer member prior to radially compressing the outer member.
A4. The method according to any one of the preceding paragraphs, wherein the outer member or the inner member are formed by pulp molding.
A5. The method according to paragraph A1, wherein the outer member and the inner member are formed by molding pulp.
A6. The method according to any one of the preceding paragraphs, wherein the outer member is a spiral wound tube.
A7. The method according to paragraph A1, wherein the outer member is a convolutely wound tube.
A8. The method according to any one of the preceding paragraphs, wherein the outer member comprises a first end region opposite the grip region.
A9. The method according to paragraph A8, comprising compressing the first end region to form an insertion region comprising two or more petals.
A10. The method according to any one of the preceding paragraphs, comprising positioning a tampon within the applicator, wherein the tampon comprises a pledget and a withdrawal member, wherein the pledget is disposed within the outer member.
A11. The method according to paragraph A2, wherein the greater than four faces of the grip region is an even number and the step of radially compressing the outer member to form the grip region is completed by applying a first pressure to the outer member to the greater than four faces that are opposite one another and at least one of the first pressure or a second pressure to the greater than four faces that are adjacent to one another, wherein the first pressure and the second pressure are different.
A12. The method according to paragraph A2, wherein the greater than four faces of the grip region is an odd number and the step of radially compressing the outer member to form the grip region is completed by applying a first pressure to the outer member to all of the greater than four faces.
B13. A method of forming a tampon applicator, the method comprising: providing an outer member comprising cellulosic material; loading the outer member into a radial compression device; radially compressing the outer member to form a grip region; and unloading the outer member comprising the grip region and a barrel region from the radial compression device.
B14. The method according to paragraph B13, wherein the grip region comprises greater than four faces and greater than four ribs, wherein each face is separated by one of the greater than four ribs.
B15. The method according to paragraph B14, comprising compressing the grip region to deform the greater than four ribs.
B16. The method according to any one of the preceding paragraphs, wherein the outer member is radially compressed by the radial compression device comprising at least two forming members configured to engage and disengage the outer member.
B17. The method according to paragraph B16, wherein each of the at least two forming members apply equal pressure to an outer surface of the outer member.
B18. The method according to paragraph B16, wherein the at least two forming members simultaneously apply pressure to the outer member to form the grip region.
B19. The method according to paragraph B14, wherein each of the greater than four ribs are formed by compressing a portion of the outer member between adjacent forming members of the radial compression device.
B20. The method according to paragraph B16, wherein the at least two forming members comprise a face-impression region and a rib-impression region.
B21. The method according to any one of the preceding paragraphs, comprising providing an applicator parent roll and cutting the applicator parent roll to form the outer member.
B22. The method according to any one of the preceding paragraphs, wherein the outer member has a first length prior to formation of the grip region and a second length after formation of the grip region, wherein the first length is greater than the second length.
B23. The method according to any one of the preceding paragraphs, comprising radially compressing a second portion of the outer member to form a second grip region.
B24. The method according to any one of the preceding paragraphs, comprising rolling an end region of the outer member.
B25. The method according to any one of the preceding paragraphs, comprising providing an inner member.
B26. The method according to paragraph B25, comprising compressing the inner member to form an engagement region, wherein the engagement region is configured to operatively engage the grip region upon the inner member being positioned in a pre-dispensing position.
B27. The method according to paragraph B26, comprising compressing the inner member to form a second engagement region, wherein the second engagement region is configured to operatively engage the grip region upon movement of the inner member to a post-dispensing position.
C28. A method of forming a tampon applicator, the method comprising: providing an outer member comprising two or more layers of material, wherein the outer member is formed by at least one of spirally winding and convolutely winding the two or more layers of material, wherein at least one of the two or more layers of material comprises cellulosic material; loading the outer member into a radial compression device; radially compressing the outer member to from a grip region; unloading the outer member comprising the grip region and a barrel region from the radial compression device.
C29. The method according to paragraph C28, comprising rolling an end region of the outer member to form a rolled edge region.
C30. The method according to any one of the preceding paragraphs, wherein the grip region comprises at least one of a first face and a second face.
C31. The method according to paragraph C30, wherein the first face comprises a first indicia and the second face comprises a second indicia.
C32. The method according to paragraph C31, wherein the first indica and the second indica are different.
C33. The method according to paragraph C29, wherein the rolled edge region is tapered.
C34. The method according to paragraph C28, wherein the outer member comprises a first end region.
C35. The method according to paragraph C34, comprising folding the first end region to form two or more pleats.
C36. The method according to paragraph C34, comprising forming at least two petals in the first end region.
C37. The method according to paragraph C28, wherein the grip region comprises greater than four faces.
C38. The method according to paragraph C37, wherein each of the greater than four faces extends from an upper grip region to a lower grip region.
C39. The method according to paragraph C38, wherein each of the greater than four faces have a radius of curvature that is about 300 mm.
C40. The method according to paragraph C38, wherein each of the greater than four faces have a radius of curvature that is from about 4.3 mm to about 900 mm.
C41. The method according to paragraph C28, wherein the tampon applicator exhibits a water content of from 0% to about 20%.
C42. The method according to paragraph C28, wherein the tampon applicator exhibits a water content of from 1% to about 15%.
C43. The method according to paragraph C28, wherein the tampon applicator exhibits a water content of from 4% to about 10%.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A method of forming a tampon applicator, the method comprising:
providing an applicator comprising an outer member and an inner member, wherein each of the outer member and the inner member comprise cellulosic material;
loading the outer member into a compression device;
compressing the outer member to form a grip region, wherein the grip region comprises greater than four faces that are circumferentially formed about the outer member; and
unloading the outer member comprising the grip region and a barrel region from the compression device.

2. The method of claim 1, wherein the compressing the outer member comprises radially compressing the outer member to form the grip region.

3. The method of claim 2, comprising inserting a stabilizing rod within an interior portion of the outer member prior to radially compressing the outer member.

4. The method of claim 1, wherein at least one of the outer member and the inner member is formed by pulp molding.

5. The method of claim 1, wherein the outer member is at least one of a spiral wound tube and a convolutely wound tube.

6. The method of claim 1, wherein the outer member comprises a first end region opposite the grip region, and wherein the first end region is compressed to form an insertion region comprising two or more petals.

7. The method of claim 1, comprising positioning a tampon within the applicator, wherein the tampon comprises a pledget and a withdrawal member, wherein the pledget is disposed within the outer member.

8. The method of claim 2, wherein the greater than four faces of the grip region is an even number and the step of radially compressing the outer member to form the grip region is completed by applying a first pressure to the outer member to the greater than four faces that are opposite one another and at least one of the first pressure or a second pressure to the greater than four faces that are adjacent to one another, wherein the first pressure and the second pressure are different.

9. The method of claim 2, wherein the greater than four faces of the grip region is an odd number and the step of radially compressing the outer member to form the grip region is completed by applying a first pressure to the outer member to all of the greater than four faces.

10. A method of forming a tampon applicator, the method comprising:
providing an outer member comprising cellulosic material;
loading the outer member into a radial compression device;
radially compressing the outer member to form a grip region,
unloading the outer member comprising the grip region and a barrel region from the radial compression device.

11. The method of claim 10, wherein the grip region comprises greater than four faces and greater than four ribs, wherein each face is separated by one of the greater than four ribs.

12. The method of claim 11, comprising compressing the grip region to deform the greater than four ribs.

13. The method of claim 10, wherein the outer member is radially compressed by the radial compression device comprising at least two forming members configured to engage and disengage the outer member, wherein each of the at least two forming members apply equal pressure to an outer surface of the outer member.

14. The method of claim 13, wherein the at least two forming members simultaneously apply pressure to the outer member to form the grip region.

15. The method of claim 11, wherein each of the greater than four ribs are formed by compressing a portion of the outer member between adjacent forming members of the radial compression device.

16. The method of claim 13, wherein the at least two forming members comprise a face-impression region and a rib-impression region.

17. The method of claim 10, comprising providing an applicator parent roll and cutting the applicator parent roll to form the outer member.

18. The method of claim 10, wherein the outer member has a first length prior to formation of the grip region and a second length after formation of the grip region, wherein the first length is greater than the second length.

19. The method of claim 10, comprising radially compressing a second portion of the outer member to form a second grip region; and rolling an end region of the outer member.

20. The method of claim 10, comprising providing an inner member; compressing the inner member to form an engagement region, wherein the engagement region is configured to operatively engage the grip region upon the inner member being positioned in a pre-dispensing position; and compressing the inner member to form a second engagement region, wherein the second engagement region is configured to operatively engage the grip region upon movement of the inner member to a post-dispensing position.

21. A method of forming a tampon applicator, the method comprising:
providing an outer member comprising two or more layers of material, wherein the outer member is formed by at least one of spirally winding and convolutely winding the two or more layers of material, wherein at least one of the two or more layers of material comprises cellulosic material;
loading the outer member into a radial compression device;
radially compressing the outer member to from a grip region;
unloading the outer member comprising the grip region and a barrel region from the radial compression device.
